SEQUENCE LISTING (39 sequences)

SEQ ID NO: 1 - protein, 34 aa, Artificial Sequence
  Description of Artificial Sequence Synthetic peptide
IKPEAPGEDASPEELNRYYASLRHYLNLVTRQRY

SEQ ID NO: 2 - protein, 15 aa, Artificial Sequence
  Description of Artificial Sequence Synthetic peptide
IKXRHYLNLVTRQRY

SEQ ID NO: 3 - protein, 15 aa, Artificial Sequence
  Description of Artificial Sequence Synthetic peptide
IKXRHYLNLVTRQRY

SEQ ID NO: 4 - protein, 15 aa, Artificial Sequence
  Description of Artificial Sequence Synthetic peptide
IKXRHYLNLVTRQRY

SEQ ID NO: 5 - protein, 15 aa, Artificial Sequence
  Description of Artificial Sequence Synthetic peptide
IKXRHYLNLVTRQRY

SEQ ID NO: 6 - protein, 15 aa, Artificial Sequence
  Description of Artificial Sequence Synthetic peptide
IKXRHYLNLVTRQRY

SEQ ID NO: 7 - protein, 15 aa, Artificial Sequence
  Description of Artificial Sequence Synthetic peptide
IKXRHYLNLVTRQRY

SEQ ID NO: 8 - protein, 15 aa, Artificial Sequence
  Description of Artificial Sequence Synthetic peptide
IKXRHYLNLVTRQRY

SEQ ID NO: 9 - protein, 15 aa, Artificial Sequence
  Description of Artificial Sequence Synthetic peptide
IKXRHYLNLVTRQRF

SEQ ID NO: 10 - protein, 15 aa, Artificial Sequence
  Description of Artificial Sequence Synthetic peptide
IKXRHYLNLVTRQRF

SEQ ID NO: 11 - protein, 15 aa, Artificial Sequence
  Description of Artificial Sequence Synthetic peptide
IKXRHYLNLVTRQRF

SEQ ID NO: 12 - protein, 15 aa, Artificial Sequence
  Description of Artificial Sequence Synthetic peptide
IKXRHYLNLVTRQRF

SEQ ID NO: 13 - protein, 15 aa, Artificial Sequence
  Description of Artificial Sequence Synthetic peptide
IKXRHYLNLVTRQRF

SEQ ID NO: 14 - protein, 15 aa, Artificial Sequence
  Description of Artificial Sequence Synthetic peptide
IKXRHYLNLVTRQRF

SEQ ID NO: 15 - protein, 15 aa, Artificial Sequence
  Description of Artificial Sequence Synthetic peptide
IKXRHYLNLVTRQRF

SEQ ID NO: 16 - protein, 15 aa, Artificial Sequence
  Description of Artificial Sequence Synthetic peptide
IKXRHYLNLVTRQRF

SEQ ID NO: 17 - protein, 15 aa, Artificial Sequence
  Description of Artificial Sequence Synthetic peptide
IKXRHYLNLVTRQRF

SEQ ID NO: 18 - protein, 15 aa, Artificial Sequence
  Description of Artificial Sequence Synthetic peptide
IKXRHYLNLVTRQRX

SEQ ID NO: 19 - protein, 15 aa, Artificial Sequence
  Description of Artificial Sequence Synthetic peptide
IKXRHYLNLVTRQRW

SEQ ID NO: 20 - protein, 15 aa, Artificial Sequence
  Description of Artificial Sequence Synthetic peptide
IKXRHYLNLVTRQRX

SEQ ID NO: 21 - protein, 15 aa, Artificial Sequence
  Description of Artificial Sequence Synthetic peptide
IKXRHYLNLVTRQRX

SEQ ID NO: 22 - protein, 15 aa, Artificial Sequence
  Description of Artificial Sequence Synthetic peptide
IKXRHYLNLVTRQRY

SEQ ID NO: 23 - protein, 15 aa, Artificial Sequence
  Description of Artificial Sequence Synthetic peptide
IKXRHYLNWVTRQRY

SEQ ID NO: 24 - protein, 15 aa, Artificial Sequence
  Description of Artificial Sequence Synthetic peptide
IXXRHYLNWVTRQRY

SEQ ID NO: 25 - protein, 15 aa, Artificial Sequence
  Description of Artificial Sequence Synthetic peptide
IKXRHYLNWVTRQRY

SEQ ID NO: 26 - protein, 15 aa, Artificial Sequence
  Description of Artificial Sequence Synthetic peptide
IKXRHYLNWVTRQRY

SEQ ID NO: 27 - protein, 15 aa, Artificial Sequence
  Description of Artificial Sequence Synthetic peptide
IKXRHYLNWVTRQRY

SEQ ID NO: 28 - protein, 15 aa, Artificial Sequence
  Description of Artificial Sequence Synthetic peptide
IKXRHYLNWVTRQRY

SEQ ID NO: 29 - protein, 15 aa, Artificial Sequence
  Description of Artificial Sequence Synthetic peptide
IKXRHYLNWVTRQRY

SEQ ID NO: 30 - protein, 15 aa, Artificial Sequence
  Description of Artificial Sequence Synthetic peptide
IKXRHYLNWVTRQRY

SEQ ID NO: 31 - protein, 15 aa, Artificial Sequence
  Description of Artificial Sequence Synthetic peptide
IKXRHYLNWVTRQRY

SEQ ID NO: 32 - protein, 26 aa, Homo sapiens
GIGAVLKVLTTGLPALISWIKRKRQQ

SEQ ID NO: 33 - protein, 39 aa, Homo sapiens
FALLGDFFRKSKEKIGKEFKRIVQRIKDFLRNLVPRTES

SEQ ID NO: 34 - protein, 39 aa, Homo sapiens
NKRFALLGDFFRKSKEKIGKEFKRIVQRIKDFLRNLVPR

SEQ ID NO: 35 - protein, 30 aa, Homo sapiens
QHRYQQLGAGLKVLFKKTHRILRRLFNLAK

SEQ ID NO: 36 - protein, 107 aa, Mus musculus
DVQMTQSTSSLSASLGDRVTISCRASQDIKNYLNWYQQKPGGTVKLLIYYSSTLLSGVPSRFSGRGSGTDFSLTITNLEREDIATYFCQQSITLPPTFGGGTKLEIK

SEQ ID NO: 37 - protein, 125 aa, Mus musculus
EVQLVESGGGLVKPGGSLKLSCAVSGFTFSDYAMSWIRQTPENRLEWVASINIGATYAYYPDSVKGRFTISRDNAKNTLFLQMSSLGSEDTAMYYCARPGSPYEYDKAYYSMAYWGPGTSVTVSS

SEQ ID NO: 38 - protein, 108 aa, Artificial Sequence
  humanized <dig> VL
DIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKR

SEQ ID NO: 39 - protein, 125 aa, Artificial Sequence
  Humanized <dig> VH
QVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSS

(19) United States
(12) Patent Application Publication
(10) Pub. No.: US 2013/0280279 A1
(43) Pub. Date: Oct. 24, 2013

(54) PHARMACEUTICAL COMPOSITION OF A COMPLEX OF AN ANTI-DIG ANTIBODY AND DIGOXIGENIN THAT IS CONJUGATED TO A PEPTIDE

(71) Applicant: HOFFMANN-LA ROCHE INC., NUTLEY, NJ (US)

(72) Inventors: Ulrich Brinkmann, Weilheim (DE); Sebastian Dziadek, Benediktbeuern (DE); Eike Hoffmann, Herrsching a. Ammersee (DE)

(21) Appl. No.: 13/929,231

(22) Filed: Jun. 27, 2013

Related U.S. Application Data
(63) Continuation of application No. PCT/EP2011/074273, filed on Dec. 30, 2011.

(30) Foreign Application Priority Data
Jan. 3, 2011 (EP) 11150037.7

(51) Int. Cl. A61K 47/48

(52) U.S. Cl. CPC A61K 47/48692
USPC 424/175.1; 530/391.9

(57) ABSTRACT

The present invention relates to a pharmaceutical composition of complex of a monospecific antibody that binds to digoxigenin, and a digoxigenin-conjugated peptide, to the isolated or recovered complex as well as to a method of producing such complex or composition. Furthermore the use of such a pharmaceutical composition as a medicament is described.

### CROSS-REFERENCE TO RELATED APPLICATIONS

This application is a continuation of International Application No. PCT/EP2011/074273 having an international filing date of Dec. 30, 2011, the entire contents of which are incorporated herein by reference, and which claims benefit under 35 U.S.C. §119 to European Patent Application No. 11150037.7, filed Jan. 3, 2011.

### SEQUENCE LISTING

The instant application contains a Sequence Listing submitted via EFS-Web and hereby incorporated by reference in its entirety. Said ASCII copy, created on Jun. 3, 2013, is named P4579C1SeqList.txt, and is 19,886 bytes in size.

### FIELD OF THE INVENTION

The present invention relates to a pharmaceutical composition of complex of a monospecific antibody that binds to digoxigenin, and a digoxigenin-conjugated peptide, to the recovered complex as well as to a method of producing such complex or composition. Furthermore the use of such a pharmaceutical composition as a medicament is described.

### BACKGROUND OF THE INVENTION

U.S. Pat. No. 5,804,371 relates to hapten-labelled peptides and their use in an immunological method of detection.

WO 2006/094269 and WO 2009/136352 relate to antiangiogenic compounds, to VEGF binding peptides and macromolecules incorporating these peptides.

WO 2006/095166 relates to modified PYY (3-36) peptides and their effects on feeding behavior.

WO 2007/065808 relates to Neuropeptide-2 Receptor agonists and PYY derivatives and their use for the treatment of diseases such as obesity and diabetes.

Decarie A., et al, Peptides, 15 (1994) 511-518, relates to a digoxogenin-labeled peptide (Bradykinin) and its application to chemiluminoenzyme immunoassay of Bradykinin in inflamed tissues. No isolated or recovered complex of a digoxogenin-labeled peptide and an anti-DIG antibody is described. Also nor pharmaceutical composition or use of such complex is described.

### SUMMARY OF THE INVENTION

One aspect of the invention is a pharmaceutical composition comprising a complex of:

a) a monospecific antibody that binds to digoxigenin, and

b) digoxigenin wherein the digoxigenin is conjugated to a peptide consisting of 5 to 60 amino acids.

Another aspect of the invention is a complex of:
a) a monospecific antibody that binds to digoxigenin, and
b) digoxigenin wherein the digoxigenin is conjugated to a peptide consisting of 5 to 60 amino acids,
wherein the complex has been recovered after production. In one embodiment the antibody of a) is a monoclonal antibody.
In one embodiment the antibody of a) comprises a heavy chain variable domain of SEQ ID NO:37 and a light chain variable domain of SEQ ID NO:36.

In one embodiment the antibody of a) is a humanized or human antibody.
In one embodiment the antibody of a) comprises a heavy chain variable domain of SEQ ID NO:39 and a light chain variable domain of SEQ ID NO:38.

In one embodiment the peptide is selected from the group consisting of:

(SEQ ID NO: 26)
Ac-IK-Pqa-RHYLNWVTRQ(N-methyl)RY;
(SEQ ID NO: 32)
GIGAVLKVLTTGLPALISWIKRKRQQ;
(SEQ ID NO: 33)
FALLGDFFRKSKEKIGKEFKRIVQRIKDFLRNLVPRTES;
(SEQ ID NO: 34)
NKRFALLGDFFRKSKEKIGKEFKRIVQRIKDFLRNLVPR;
and
(SEQ ID NO: 35)
QHRYQQLGAGLKVLFKKTHRILRRLFNLAK.

One embodiment is the pharmaceutical composition or the complex according to the invention for the treatment of metabolic diseases.
One embodiment is the pharmaceutical composition or the complex according to the invention for the treatment of cancer.
One embodiment is the pharmaceutical composition or the complex according to the invention for the treatment of inflammatory diseases.
One embodiment is a method of producing a complex according to the invention comprising the steps of
   complexation of the monospecific antibody that binds to digoxigenin, and digoxigenin wherein the digoxigenin is conjugated to a peptide consisting of 5 to 60 amino acids
   recovering of the resulting complex.

The pharmaceutical compositions and complexes according to the invention show valuable properties like good in vivo serum half-life (as compared to the parent peptides) and they have high biological activity. They are therefore especially useful as peptide based medicaments with a defined structure.

### DESCRIPTION OF THE FIGURES

FIG. 1: Schematic model of humanized <Dig> IgG

FIG. 2: Procedure for digoxigenation (conjugation of digoxigenin to) of peptides (see e.g. FIG. 2A) and examples of the digoxigenated fluorophore Dig-Cy5 (FIG. 2a, the fluorophore was used as analytical surrogate for the peptide) and of the digoxigenated PYY-derivative DIG-moPYY (DIG-moPYY) (FIG. 2C):

FIG. 3: Exemplary scheme of a complex of a monospecific digoxigenin binding anti-DIG antibody and bispecific anti-DIG antibody with digoxigenin which conjugated to a peptide or to fluorophore

FIG. 4: Proof of concept: complexes of anti-DIG antibodies (bispecifics are used for proof of concept) with digoxigenated fluorophore (as analytical surrogate for peptides) Cy5: Size exclusion chromatography of digoxigenated Cy5 <Her2>-<Dig> bispecific antibody complex indicates charging with digoxigenated Cy5 and homogeneity of charged molecules. A chromatogram: 1: Her2 Dig Cy5 (1:0) 2: Her2 Dig Cy5 (1:0.5), 3: Her2 Dig Cy5 (1:1), 4: Her2 Dig Cy5 (1:2), 5: Her2 Dig Cy5 (1:3), 6: Her2 Dig Cy5 (1:5). 7: Her2 Dig Cy5 (0:1), B analysis: Charging of bivalent digoxigenin-binding antibodies becomes saturated at a 2:1 payload:antibody ratio.

FIG. 5: Complex of anti-DIG antibody with digoxigenin which conjugated to a peptide: Antibody complexation of digoxigenin which conjugated to a peptide results in a complex of defined size as demonstrated by size exclusion chromatography.

FIG. 6: Charging of anti-DIG antibody with digoxigenin which conjugated to a peptide: SEC-MALLS analyses demonstrate that antibody complexation of digoxigenated peptides result in a complex of defined size which is larger than uncomplexed antibody or uncomplexed peptide and contains 2 peptides per antibody derivative.

FIG. 7: Improved biological activity of digoxigenated and antibody-complexed peptide compared to PEGylated peptide in vitro

FIG. 8: Improved in vivo serum half-life/stability of a digoxigenated fluorescent dye (as surrogate for peptide) upon antibody complexation.

FIG. 9: Improved in vivo serum half-life/stability of a digoxigenated peptide upon antibody complexation.

FIG. 10: Improved in vivo activity of antibody-complexed digoxigenated peptides compared to uncomplexed peptides. In vivo potency of the IgG-complexed DIG-moPYY-peptide can be detected by reduction in food intake in treated animals.

FIG. 11: Improved in vivo activity of antibody-complexed digoxigenated peptides compared to uncomplexed peptides. In vivo potency of the IgG-complexed DIG-moPYY-peptide can be detected the differences of food intake in animals that received uncomplexed peptides compared to animals that received a 17-fold lower dose of complexed peptide.

### DETAILED DESCRIPTION OF THE INVENTION

One aspect of the invention is a pharmaceutical composition comprising a complex of:
   a) a monospecific antibody that binds to digoxigenin, and
   b) digoxigenin wherein the digoxigenin is conjugated to a peptide consisting of 5 to 60 amino acids.

Another aspect of the invention a complex of:
a) a monospecific antibody that binds to digoxigenin, and
b) digoxigenin wherein the digoxigenin is conjugated to a peptide consisting of 5 to 60 amino acids.

In one embodiment the peptide comprises 10 to 50 amino acids. Peptides with 12 or more amino acids typically have a secondary structure. Therefore in one embodiment the peptide comprises 12 to 40 amino acids. In one embodiment the peptide comprises 12 to 30 amino acids.

The terms “digoxigenin” or “digoxygenin” or “DIG” are used interchangeable herein and refer to 3-[(3S,5R,8R,9S,10S,12R,13S,14S,17R)-3,12,14-trihydroxy-10,13-dimethyl-1,2,3,4,5,6,7,8,9,11,12,15,16,17-tetradecahydro-cyclopenta[a]-phenanthren-17-yl]-2H-furan-5-one (CAS number 1672-46-4). Digoxigenin (DIG) is a steroid found exclusively in the flowers and leaves of the plants Digitalis purpurea, Digitalis orientalis and Digitalis lanata (foxgloves) (Polya, G., Biochemical targets of plant bioactive compounds, CRC Press, New York (2003) p. 847).

The terms “anti-digoxigenin antibody” and “an antibody that binds to digoxigenin” refer to an antibody that is capable of binding digoxigenin with sufficient affinity such that a complex of a) a monospecific antibody that binds to digoxigenin, and b) digoxigenin wherein the digoxigenin is conjugated to a peptide consisting of 5 to 60 amino acids, is formed which is useful as a therapeutic agent prolonging the half-time of the peptide.

The term “a digoxigenin that is conjugated to therapeutic peptide” refers to a digoxigenin which is covalently linked to a peptide. Typically the digoxigenin is conjugate via its 3-hydroxy group to the peptide. Activated Digoxigenin-3-carboxy-methyl derivatives are often used as starting materials for such conjugated digoxigenin peptides. In one embodiment the digoxigenin is conjugated (preferably via its 3-hydroxy group) to the peptide via a linker. Said linker can comprise a) a methylene-carboxy-methyl group (—CH2-C(O)—), b) from 1 to 10 (preferably from 1 to 5) amino acids (e.g. selected from glycine, serine, glutamate, β-alanine, γ-aminobutyric acid, β-aminocaproic acid or lysine) and/or c) one or more (preferably one or two) compounds having the structural formula NH2-[(CH2)nO]xCH2-CH2-COOH in which n is 2 or 3 and x is 1 to 10, preferably 1 to 7 (which results (at least partly) in a linker (part) of the formula —NH-[(CH2)nO]xCH2-CH2-C(O)—; one example of such a compound is e.g. 12-amino-4,7,10-trioxadodecanoic acid (results in a TEG (Triethylenglycol) linker or TEG spacer, see Example 5)). In one embodiment the linker further comprises a maleimido group. Examples of digoxigenin conjugated to a peptide via such linkers are described in the Example 5 below. The linker has a stabilizing and solubilizing effect since it preferably contains charges or/and can form hydrogen bridges. In addition it can sterically facilitate the binding of the anti-digoxigenin antibody to the digoxigenin-conjugated peptide. In one embodiment the linker is located at a side chain of an amino acid of the peptide (e.g. conjugated to a lysine or cystein side chain via the amino or thio group). In one embodiment the linker is located at the amino terminus or at the carboxy terminus of the peptide. The position of the linker on the peptide is typically chosen at a region where the biological activity of the peptide is not affected. Therefore the attachment position of the linkers depends on the nature of the peptide and the relevant structure elements which are responsible for the biological activity. The biological activity of the peptide to which the digoxigenin attached can be tested in an in vitro assay.

The term “peptide,” as used herein refers to a polymer of amino acids. As used herein, these terms apply to amino acid polymers in which one or more amino acid residues is an artificial chemical analog of a corresponding naturally occurring amino acid. These terms also apply to naturally occurring amino acid polymers. Amino acids can be in the L or D form. Peptides may be cyclic, having an intramolecular bond between two non-adjacent amino acids within the peptide, e.g., backbone to backbone, side-chain to backbone and side-chain to side-chain cyclization. Cyclic peptides can be prepared by methods well know in the art. See e.g., U.S. Pat. No. 6,013,625. Typical biologically active peptides are described e.g. in Bellmann-Sickert, K., et al., Trends Pharm. Sci. 31 (2010) 434-441.

All peptide sequences are written according to the generally accepted convention whereby the alpha-N-terminal amino acid residue is on the left and the alpha-C-terminal amino acid residue is on the right. As used herein, the term “N-terminus” refers to the free alpha-amino group of an amino acid in a peptide, and the term “C-terminus” refers to the free a-carboxylic acid terminus of an amino acid in a peptide. A peptide which is N-terminated with a group refers to a peptide bearing a group on the alpha-amino nitrogen of the N-terminal amino acid residue. An amino acid which is N-terminated with a group refers to an amino acid bearing a group on the alpha-amino nitrogen.

Unless indicated otherwise by a “D” prefix, e.g., D-Ala or N-Me-D-Ile, or written in lower case format, e.g., a, i, l, (D versions of Ala, Ile, Leu), the stereochemistry of the alpha-carbon of the amino acids and aminoacyl residues in peptides described in this specification and the appended claims is the natural or “L” configuration. The Cahn-Ingold-Prelog “R” and “S” designations are used to specify the stereochemistry of chiral centers in certain acyl substituents at the N-terminus of the peptides. The designation “R,S” is meant to indicate a racemic mixture of the two enantiomeric forms. This nomenclature follows that described in Cahn, R. S., et al., Angew. Chem. Int. Ed. Engl. 5 (1966) 385-415.

In general, the term “amino acids” as used herein refers to natural an non-natural amino acids and their derivatives. Examples of such amino acids include, but are not limited to, Aad (alpha-Aminoadipic acid), Abu (Aminobutyric acid), Ach (alpha-aminocyclohexane-carboxylic acid), Acp (alpha-aminocyclopentane-carboxylic acid), Acpc (1-Aminocyclopropane-1-carboxylic acid), Aib (alpha-aminoisobutyric acid), Aic (2-Aminoindane-2-carboxylic acid; also called 2-2-Aic), 1-1-Aic (1-aminoindane-1-carboxylic acid), (2-aminoindane-2-carboxylic acid), Ala, Allylglycine (AllylGly), Alloisoleucine (allo-Ile), Arg, Asn, Asu (alpha-Aminosuberic acid, 2-Aminooctanedioc acid), Asp, Bip (4-phenyl-phenylalanine-caroxylic acid), BnHP ((2S,4R)-4-Hydroxyproline), Cha (beta-cyclohexylalanine), Cit (Citrulline), Cyclohexylglycine (Chg), Cyclopentylalanine, beta-Cyclopropyl alanine, Cys, Dab (1,4-Diaminobutyric acid), Dap (1,3-Diaminopropionic acid), p (3,3-diphenylalanine-carboxylic acid), 3,3-Diphenylalanine, Di-n-propylglycine (Dpg), 2-Furylalanine, Gln, Glu, Gly, His, Homocyclohexylalanine (HoCha), Homocitrulline (HoCit), Homocycloleucine, Homoleucin (HoLeu), Homoarginine (HoArg), Homoserine (HoSer), Hydroxyproline, Ile, Leu, Lys, Lys (Ac), (1) Nal (1-Naphtyl Alanine), (2) Nal (2-Naphtyl Alanine), Met, 4-MeO-Apc (1-amino-4-(4-methoxyphenyl)-cyclohexane-1-carboxylic acid), Nor-leucine (Nle), Nva (Norvaline), Omathine, 3-Pal (alpha-amino-3-pyridylalanine-carboxylic acid), 4-Pal (alpha-amino-4-pyridylalanine-carboxylic acid), Phe, 3,4,5,F3-Phe (3,4,5-Trifluoro-phenylalanine), 2,3,4,5,6,F5-Phe (2,3,4,5,6-Pentafluoro-phenylalanine), Pqa (4-oxo-6-(1-piperazinyl)-3 (4H)-quinazoline-acetic acid (CAS 889958-08-1)), Pro, Pyridylalanine, Quinolylalanine, Ser, Sarcosine (Sar), Thiazolylalanine, Thienylalanine, Thr, Tic (alpha-amino-1,2,3,4,tetrahydroisoquinoline-3-carboxylic acid), Tic (OH), Tle (tertbutylGlycine), Trp, Tyr, Tyr (Me), Val.

In one embodiment of the invention the amino acid is selected from the group consisting of the list above.

For convenience in describing this invention, the abbreviations for the natural amino acids are listed below:

Asp=D=Aspartic Acid; Ala=A=Alanine; Arg=R=Arginine; Asn=N=Asparagine; Gly=G=Glycine; Glu=E=Glutamic Acid; Gln=Q=Glutamine; His=H=Histidine; Ile=1=Isoleucine; Leu=L=Leucine; Lys=K=Lysine; Met=M=Methionine; Phe=F=Phenylalanine; Pro=P=Proline; Ser=S=Serine; Thr=T=Threonine; Trp=W=Tryptophan; Tyr=Y=Tyrosine; Cys=C=Cysteine; and Val=V=Valine.

A non-limiting list of abbreviations for some of the typical amino acids derivatizations is shown below:

Ac=Acetyl; Boc=9-Fluorenylmethoxycarbonyl; Dde=; Fmoc=9-Fluorenylmethoxycarbonyl; Mtr=4-Methoxy-2,3,6-trimethylbenzenesulfonyl; Pbf=2,2,4,6,7-Pentamethyldihydrobenzofuran-5-sulfonyl; Trt=Trityl, tBu=tert-Butyl; TEG=4,7,10-trioxadodecanoic acid (=Triethylenglycol (TEG)-linker).

In one embodiment the peptide is a neuropeptide-2 receptor agonist as described e.g. WO 2007/065808. In one embodiment the peptide is selected from the group consisting of

(SEQ ID NO: 2)
IK-Pqa-RHYLNLVTRQRY;
(SEQ ID NO: 3)
IK-Pqa-RHYLNLVTRQ(N-methyl)RY;
(SEQ ID NO: 4)
IK-Pqa-RHYLNLVTRQ(N-methyl)R(m-)Y;
(SEQ ID NO: 5)
IK-Pqa-RHYLNLVTRQ(N-methyl)R(3-I)Y;
(SEQ ID NO: 6)
IK-Pqa-RHYLNLVTRQ(N-methyl)R(3-5 di F)Y;
(SEQ ID NO: 7)
IK-Pqa-RHYLNLVTRQ(N-methyl)R(2-6 di F)Y;
(SEQ ID NO: 8)
IK-Pqa-RHYLNLVTRQ(N-methyl)R(2-6 di Me)Y;
(SEQ ID NO: 9)
IK-P qa-RHYLNLVTRQ(N-methyl)RF(O—CH3);
(SEQ ID NO: 10)
IK-Pqa-RHYLNLVTRQ(N-methyl)RF;
(SEQ ID NO: 11)
IK-Pqa-RHYLNLVTRQ(N-methyl)R(4-NH2)Phe;
(SEQ ID NO: 12)
IK-Pqa-RHYLNLVTRQ(N-methyl)R(4-F)Phe;
(SEQ ID NO: 13)
IK-Pqa-RHYLNLVTRQ(N-methyl)R(4-CH2OH)Phe;
(SEQ ID NO: 14)
IK-Pqa-RHYLNLVTRQ(N-methyl)R(4-CF3)Phe;
(SEQ ID NO: 15)
IK-Pqa-RHYLNLVTRQ(N-methyl)R(3-F)Phe;
(SEQ ID NO: 16)
IK-Pqa-RHYLNLVTRQ(N-methyl)R(2,3.4,5,6-Penta-F)
Phe;
(SEQ ID NO: 17)
IK-Pqa-RHYLNLVTRQ(N-methyl)R(3.4-diC1)Phe;
(SEQ ID NO: 18)
IK-Pqa-RHYLNLVTRQ(N-methyl)RCha;
(SEQ ID NO: 19)
IK-Pqa-RHYLNLVTRQ(N-methyl)RW;
(SEQ ID NO: 20)
IK-Pqa-RHYLNLVTRQ(N-methyl)R(1)Nal;
(SEQ ID NO: 21)
IK-Pqa-RHYLNLVTRQ(N-methyl)R(2)Nal;
(SEQ ID NO: 22)
IK-Pqa-RHYLNLVTRQR-C-α-Me-Tyr;
(SEQ ID NO: 23)
IK-Pqa-RHYLNWVTRQ(N-methyl)RY;
(SEQ ID NO: 24)
INle-Pqa-RHYLNWVTRQ(N-methyl)RY;
(SEQ ID NO: 25)
Ac-IK-Pqa-RHYLNWVTRQ(N-methyl)R(2-6 di F)Y;
(SEQ ID NO: 26)
Ac-IK-Pqa-RHYLNWVTRQ(N-methyl)RY;
(SEQ ID NO: 27)
Pentyl-IK-Pqa-RHYLNWVTRQ(N-methyl)RY;
(SEQ ID NO: 28)
Trimetylacetyl-IK-Pqa-RHYLNWVTRQ(N-methyl)RY;
(SEQ ID NO: 29)
Cyclohexyl-IK-Pqa-RHYLNWVTRQ(N-methyl)RY;
(SEQ ID NO: 30)
Benzoyl-IK-Pqa-RHYLNWVTRQ(N-methyl)RY;
and
(SEQ ID NO: 31)
Adamtyl-IK-Pqa-RHYLNWVTRQ(N-methyl)RY.

In one embodiment the peptide is Ac-IK-Pqa-RHYLNWVTRQ(N-methyl)RY.

In one embodiment the peptide is Ac-IK-Pqa-RHYLNWVTRQ(N-methyl)R (2-6 di F)Y.

In one embodiment the peptide is selected from the group consisting of:

(SEQ ID NO: 26)
Ac-IK-Pqa-RHYLNWVTRQ(N-methyl)RY;
(SEQ ID NO: 32)
GIGAVLKVLTTGLPALISWIKRKRQQ;
(SEQ ID NO: 33)
FALLGDFFRKSKEKIGKEFKRIVQRIKDFLRNLVPRTES;
(SEQ ID NO: 34)
NKRFALLGDFFRKSKEKIGKEFKRIVQRIKDFLRNLVPR;
and
(SEQ ID NO: 35)
QHRYQQLGAGLKVLFKKTHRILRRLFNLAK.

In one embodiment the peptide is substantially homologous to a peptide selected from the group consisting of:

In one embodiment the peptide is selected from the group consisting of:

(SEQ ID NO: 26)
Ac-IK-Pqa-RHYLNWVTRQ(N-methyl)RY;
(SEQ ID NO: 32)
GIGAVLKVLTTGLPALISWIKRKRQQ;
(SEQ ID NO: 33)
FALLGDFFRKSKEKIGKEFKRIVQRIKDFLRNLVPRTES;
(SEQ ID NO: 34)
NKRFALLGDFFRKSKEKIGKEFKRIVQRIKDFLRNLVPR;
and
(SEQ ID NO: 35)
QHRYQQLGAGLKVLFKKTHRILRRLFNLAK.

“Substantially homologous” means at least about 85% (preferably at least about 90%, and more preferably at least about 95% or most preferably at least about 98%, of the amino-acid residues match over the defined length of the peptide sequences. Sequences that are substantially homologous can be identified by comparing the sequences using standard software available in sequence data banks, such as BLAST programs available from the National Cancer Center for Biotechnology Information at ncbi.nlm.nih.gov.

In one embodiment the peptide is characterized in that it which shows biological activity in an in vitro assay. In one embodiment the biological activity is anti-proliferative, anti-inflammatory, anti-cancer, anti-viral, or the biological activity is metabolic disease related (see e.g. Example 7).

In one embodiment the complex is characterized in that the contains non-natural amino acids. In one embodiment the complex is characterized in that the peptide that cannot be produced in living organisms.

The term “antibody” herein is used for a monospecific antibody in the broadest sense and encompasses various antibody structures, which, including but not limited to monoclonal antibodies, polyclonal antibodies, and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv).

The term “monospecific antibody that binds to digoxigenin” as used herein refers to an antibody that specifically binds only to (the cardiac glycoside) digoxigenin or derivatives thereof like e.g. digoxin, digitoxin, but that does not specifically bind to a further (distinct) antigen like e.g. a protein antigen like e.g. HER2 or IGF-1R.

The term “bispecific antibody that binds to digoxigenin” as used herein refers to an antibody that specifically binds to (the cardiac glycoside) digoxigenin or derivatives thereof like e.g. digoxin, digitoxin, and that also specifically bind to a further (distinct) antigen like e.g. a protein antigen like e.g. HER2 or IGF-1R.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and γ, respectively.

The term “complex” of a) a monospecific antibody that binds to digoxigenin, and b) digoxigenin wherein the digoxigenin is conjugated to a peptide consisting of 5 to 60 amino acids, as used herein refers to the non-covalent binding complex formed by the antibody and the digoxigenin (that is conjugated to the peptide of the invention) based on the antibody-antigen interaction.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th ed., Public Health Service, National Institutes of Health, Bethesda, Md. (1991).

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3 (L3)-FR4.

The terms “full length antibody”, “intact antibody”, and “whole antibody” are used herein interchangeably to refer to an antibody having a structure substantially similar to a native antibody structure or having heavy chains that contain an Fc region as defined herein.

The terms “host cell”, “host cell line”, and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, fifth ed., NIH Publication 91-3242, Bethesda Md. (1991), Vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3) (Chothia, C., and Lesk, A. M., J. Mol. Biol. 196 (1987) 901-917). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th ed., Public Health Service, National Institutes of Health, Bethesda, Md. (1991)). With the exception of CDR1 in VH, CDRs generally comprise the amino acid residues that form the hypervariable loops. CDRs also comprise “specificity determining residues”, or “SDRs”, which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3 (see Almagro, J. C., and Fransson, J., Front. Biosci. 13 (2008) 1619-1633). Unless otherwise indicated, HVR residues and other residues in the variable domain (e.g., FR residues) are numbered herein according to Kabat et al., supra.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs) (see, e.g., Kindt et al., Kuby Immunology, 6^{th} ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (see, e.g., Portolano, S., et al., J. Immunol. 150 (1993) 880-887; Clackson, T., et al., Nature 352 (1991) 624-628).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

### Compositions and Methods

In one aspect, the invention is based, in part, on a complex a) a monospecific antibody that binds to digoxigenin, and b) digoxigenin wherein the digoxigenin is conjugated to a peptide consisting of 5 to 60 amino acids; and a pharmaceutical composition of it. In certain embodiments, antibodies that bind to digoxigenin are provided. Antibodies of the invention are useful, e.g., for the diagnosis or treatment of cancer, metabolic or inflammatory or viral diseases.

### Exemplary Complexes of Monospecific Anti-Digoxigenin Antibodies and Digoxigenin Conjugated to a Peptide Consisting of 5 to 60 Amino Acids

In one aspect, the invention is based, in part, on a complex a) a monospecific antibody that binds to digoxigenin, and b) digoxigenin wherein the digoxigenin is conjugated to a peptide consisting of 5 to 60 amino acids.

### Antibody Affinity

As used herein, the terms “binding” or an antibody “that binds to” or “that specifically binds to” are use interchangeable and refer to the binding of the antibody to an epitope of the tumor antigen in an in vitro assay, preferably in an plasmon resonance assay (BIAcore, GE-Healthcare Uppsala, Sweden) with purified wild-type antigen. The affinity of the binding is defined by the terms ka (rate constant for the association of the antibody from the antibody/antigen complex), k_{D} (dissociation constant), and K_{D} (k_{D}/ka). Binding or specifically binding means a binding affinity (K_{D}) of 10⁻⁸ M or less, preferably 10⁻⁸ M to 10⁻¹³ M (in one embodiment 10⁻⁹ M to 10⁻¹³ M). Thus, an antibody that binds to digoxigenin according to the invention is specifically binding to digoxigenin with a binding affinity (K_{D}) of 10⁻⁸ mol/l or less, preferably 10⁻⁸ M to 10⁻¹³ M (in one embodiment 10⁻⁹ M to 10⁻¹³ M).

### Anti-Digoxigenin Antibodies

Antibodies that bind specifically to the cardiac glycosides digoxin, digitoxin, and digoxigenin can be generated e.g. as described in Hunter, M. M., et al., J. Immunol. 129 (1982) 1165-1172. One example of such antibody is the monoclonal antibody 26-10 that binds to the cardiac glycosides digoxin, digitoxin, and digoxigenin with high-affinity (KD=9 nM) (Schildbach, J. F., et al., J. Biol. Chem. 268 (1993) 21739-21747; Burks, E. A., et al., PNAS 94 (1997) 412-417).

To prepare an immunogen for immunization e.g. digoxin or digoxigenin can be conjugated to human serum albumin (Digoxin-HAS; Digoxigenin-HSA). Also Digoxigenin or digoxin-3-CMO (CMO=(O-carboxymethyl)oxime) conjugated to KLH (keyhole limpet hemocyanin) is often used. Also Digoxigenin itself can be used. Other methods to prepare digoxigenin immunogens are described e.g. in U.S. Pat. No. 4,469,797. The resulting antibodies often bind to the cardiac glycosides digoxin, digitoxin, and digoxigenin (i.e. they show cross-reactivity).

Typical antibodies that bind to digoxigenin include the monoclonal antibody 26-10, monoclonal antibody 21H8 (AbcamCat# ab420); monoclonal antibody 1.A2.1 (Santa Cruz Cat# sc-70963), monoclonal antibody (1.71.256 Roche Applied Science Cat#11333062910).

### Chimeric and Humanized Antibodies

In certain embodiments, an antibody provided herein is a chimeric antibody. Certain chimeric antibodies are described, e.g., in U.S. Pat. No. 4,816,567; and Morrison, S. L., et al., Proc. Natl. Acad. Sci. USA, 81 (1984) 6851-6855). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro, J. C., and Fransson, J., Front. Biosci. 13 (2008) 1619-1633, and are further described, e.g., in Riechmann, L., et al., Nature 332 (1988) 332-327; Queen, C., et al., Proc. Natl. Acad. Sci. USA 86 (1989) 10029-10033; U.S. Pat. Nos. 5,821,337, 7,527,791, 6,982,321, and 7,087,409; Kashmiri, S. V., et al., Methods 36 (2005) 25-34 (describing SDR (a-CDR) grafting); Padlan, E. A., Mol. Immunol. 28 (1991) 489-498 (describing “resurfacing”); Dall'Acqua, W. F., et al., Methods 36 (2005) 43-60 (describing “FR shuffling”); and Osbourn et al., Methods 36:61-68 (2005) and Klimka, A., et al., Br. J. Cancer 83 (2000) 252-260 (describing the “guided selection” approach to FR shuffling).

Human framework regions that may be used for humanization include but are not limited to: framework regions selected using the “best-fit” method (see, e.g., Sims, M. J., et al. J. Immunol. 151 (1993) 2296-2308); framework regions derived from the consensus sequence of human antibodies of a particular subgroup of light or heavy chain variable regions (see, e.g., Carter, P., et al., Proc. Natl. Acad. Sci. USA, 89 (1992) 4285-4289; and Presta, L. G., et al., J. Immunol. 151 (1993) 2623-2632); human mature (somatically mutated) framework regions or human germline framework regions (see, e.g., Almagro, J. C., and Fransson, J., Front. Biosci. 13 (2008) 1619-1633); and framework regions derived from screening FR libraries (see, e.g., Baca, M., et al., J. Biol. Chem. 272 (1997) 10678-10684 and Rosok, M. J., et al., J. Biol. Chem. 271 (1996) 22611-22618).

### Human Antibodies

In certain embodiments, an antibody provided herein is a human antibody. Human antibodies can be produced using various techniques known in the art. Human antibodies are described generally in van Dijk, M. A., and van de Winkel, J. G., Curr. Opin. Chem. Biol. 5 (2001) 368-374 and Lonberg, N., Curr. Opin. Immunol. (2008) 450-459.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, N., Nat. Biotech. 23 (2005) 1117-1125. See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described (see, e.g., Kozbor, D., J. Immunol., 133 (1984) 3001-3005; Brodeur et al., Monoclonal Antibody Production Techniques and Applications, Marcel Dekker, Inc., New York (1987), pp. 51-63; and Boerner, P., et al., J. Immunol. 147 (1991) 86-95). Human antibodies generated via human B-cell hybridoma technology are also described in Li, J., et al., Proc. Natl. Acad. Sci. USA 103 (2006) 3557-3562. Additional methods include those described, for example, in U.S. Pat. No. 7,189,826 (describing production of monoclonal human IgM antibodies from hybridoma cell lines) and Ni, J., Xiandai Mianyixue 26 (2006) 265-268 (describing human-human hybridomas). Human hybridoma technology (Trioma technology) is also described in Vollmers, H. P., and Brandlein, S., Histology and Histopathology 20 (2005) 927-937, and Vollmers, H. P., and Brandlein, S., Methods and Findings in Experimental and Clinical Pharmacology 27 (2005) 185-191.

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom, H. R., et al., Methods in Molecular Biology 178 (2001) 1-37 and further described, e.g., in McCafferty, J., et al., Nature 348, 552-554; Clackson, T., et al., Nature 352 (1991) 624-628; Marks, J. D., et al., J. Mol. Biol. 222 (1991) 581-597; Marks, J. D., and Bradbury, A., Methods in Molecular Biology 248 (2003) 161-176; Sidhu, S. S., et al., J. Mol. Biol. 338 (2004) 299-310; Lee, C. V., et al., J. Mol. Biol. 340 (2004) 1073-1093; Fellouse, F. A., Proc. Natl. Acad. Sci. USA 101 (2004) 12467-12472; and Lee, C. V., et al., J. Immunol. Methods 284 (2004) 119-132.

In certain phage display methods, repertoires of VH and VL genes are separately cloned by polymerase chain reaction (PCR) and recombined randomly in phage libraries, which can then be screened for antigen-binding phage as described in Winter, G., et al., Ann. Rev. Immunol. 12 (1994) 433-455. Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths, A. D., et al., EMBO J. 12 (1993) 725-734. Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom, H. R., and Winter, G., J. Mol. Biol., 227 (1992) 381-388. Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table below under the heading of “conservative substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; | Ile |
| | Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, P. S., Methods Mol. Biol. 207 (2008) 179-196), and/or SDRs (a-CDRs), with the resulting variant VH or VL being tested for binding affinity. Affinity maturation by constructing and reselecting from secondary libraries has been described, e.g., in Hoogenboom, H. R., et al., Methods in Molecular Biology 178 (2001) 1-37). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham, B. C., and Wells, J. A., Science 244 (1989) 1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### Recombinant Methods and Compositions

Antibodies may be produced using recombinant methods and compositions, e.g., as described in U.S. Pat. No. 4,816,567. In one embodiment, isolated nucleic acid encoding an anti-digoxigenin antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-digoxigenin antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-digoxigenin, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523 (see also Charlton, K. A., Methods in Molecular Biology 248 (2004) 245-254, describing expression of antibody fragments in E. coli). After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern (see Gerngross, T. U., Nat. Biotech. 22 (2004) 1409-1414; and L1, H., et al., Nat. Biotech. 24 (2006) 210-215).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham, F. L., et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather, J. P., et al., Annals N.Y. Acad. Sci. 383 (1982) 44-68; MRC 5 cells; and FS4 cells. Other useful mammalian host cell lines include Chinese hamster ovary (CHO) cells, including DHFR⁻ CHO cells (Urlaub, G., et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. J., and Wu, A. M., Methods in Molecular Biology 248 (2004) 255-268.

### Assays

Anti-digoxigenin antibodies provided herein may be identified, screened for, or characterized for their physical/chemical properties and/or biological activities by various assays known in the art.

### Pharmaceutical Formulations

Pharmaceutical formulations of a complex a) a monospecific antibody that binds to digoxigenin, and b) digoxigenin wherein the digoxigenin is conjugated to a peptide consisting of 5 to 60 amino acids, as described herein are prepared by mixing such antibody having the desired degree of purity with one or more optional pharmaceutically acceptable carriers (Remington's Pharmaceutical Sciences, 16th edition, Osol, A. (ed.), (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. (ed.) (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

One aspect of the invention is a pharmaceutical composition according to the invention for the treatment of metabolic diseases.

Another aspect is a pharmaceutical composition according to the invention for the treatment of cancer.

Another aspect is a pharmaceutical composition according to the invention for the treatment of inflammatory diseases.

One further aspect of the invention is a complex according to the invention for the treatment of metabolic diseases.

Another aspect is a complex according to the invention for the treatment of cancer.

Another aspect is a complex according to the invention for the treatment of inflammatory diseases.

One further aspect of the invention is a complex according to the invention for the manufacture of a medicament for the treatment of metabolic diseases.

Another aspect is a complex according to the invention for the manufacture of a medicament for the treatment of cancer.

Another aspect is a complex according to the invention for the manufacture of a medicament for the treatment of inflammatory diseases.

Another aspect of the invention is a method of treatment of a patient suffering from a metabolic disease, cancer or a inflammatory disease, by administering an effective amount of a complex according to the invention to said patient in the need of such treatment.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Sequence Listing

| SEQ ID NO: 1 | PYY 3-36: |
|---|---|
| | IKPEAPGEDASPEELNRYYASLRHYLNLVTRQRY |
| | (3-36) |
| | PYY derivatives |
| SEQ ID NO: 2 | IK-Pqa-RHYLNLVTRQRY |
| SEQ ID NO: 3 | IK-Pqa-RHYLNLVTRQ(N-methyl)RY |
| SEQ ID NO: 4 | IK-Pqa-RHYLNLVTRQ(N-methyl)R(m-)Y |
| SEQ ID NO: 5 | IK-Pqa-RHYLNLVTRQ(N-methyl)R(3-I)Y |
| SEQ ID NO: 6 | IK-Pqa-RHYLNLVTRQ(N-methyl)R(3-5 di F)Y |
| SEQ ID NO: 7 | IK-Pqa-RHYLNLVTRQ(N-methyl)R(2-6 di F)Y |
| SEQ ID NO: 8 | IK-Pqa-RHYLNLVTRQ(N-methyl)R(2-6 di Me)Y |
| SEQ ID NO: 9 | IK-Pqa-RHYLNLVTRQ(N-methyl)RF(O—CH3) |
| SEQ ID NO: 10 | IK-Pqa-RHYLNLVTRQ(N-methyl)RF |
| SEQ ID NO: 11 | IK-Pqa-RHYLNLVTRQ(N-methyl)R(4-NH2)Phe |
| SEQ ID NO: 12 | IK-Pqa-RHYLNLVTRQ(N-methyl)R(4-F)Phe |
| SEQ ID NO: 13 | IK-Pqa-RHYLNLVTRQ(N-methyl)R(-CH2OH)Phe |
| SEQ ID NO: 14 | IK-Pqa-RHYLNLVTRQ(N-methyl)R(-CF3)Phe |
| SEQ ID NO: 15 | IK-Pqa-RHYLNLVTRQ(N-methyl)R(-F)Phe |
| SEQ ID NO: 16 | IK-Pqa-RHYLNLVTRQ(N-methyl)R(2,3.4,5,6-Penta-F)Phe |
| SEQ ID NO: 17 | IK-Pqa-RHYLNLVTRQ(N-methyl)R(3.4-diC1)Phe |
| SEQ ID NO: 18 | IK-Pqa-RHYLNLVTRQ(N-methyl)RCha |
| SEQ ID NO: 19 | IK-Pqa-RHYLNLVTRQ(N-methyl)RW |
| SEQ ID NO: 20 | IK-Pqa-RHYLNLVTRQ(N-methyl)R(1)Nal |
| SEQ ID NO: 21 | IK-Pqa-RHYLNLVTRQ(N-methyl)R(2)Nal |
| SEQ ID NO: 22 | IK-Pqa-RHYLNLVTRQR-C-α-Me-Tyr |
| SEQ ID NO: 23 | IK-Pqa-RHYLNWVTRQ(N-methyl)RY |
| SEQ ID NO: 24 | INle-Pqa-RHYLNWVTRQ(N-methyl)RY |
| SEQ ID NO: 25 | Ac-IK-Pqa-RHYLNWVTRQ(N-methyl)R(2-6 di F)Y |
| SEQ ID NO: 26 | Ac-IK-Pqa-RHYLNWVTRQ(N-methyl)RY(= moPYY) |
| SEQ ID NO: 27 | Pentyl-IK-Pqa-RHYLNWVTRQ(N-methyl)RY |
| SEQ ID NO: 28 | Trimetylacetyl-IK-Pqa-RHYLNWVTRQ(N-methyl)RY |
| SEQ ID NO: 29 | Cyclohexyl-IK-Pqa-RHYLNWVTRQ(N-methyl)RY |
| SEQ ID NO: 30 | Benzoyl-IK-Pqa-RHYLNWVTRQ(N-methyl)RY |
| SEQ ID NO: 31 | Adamtyl-IK-Pqa-RHYLNWVTRQ(N-methyl)RY |
| | Peptides with antitumor effect |
| SEQ ID NO: 32 | GIGAVLKVLTTGLPALISWIKRKRQQ |
| SEQ ID NO: 33 | FALLGDFFRKSKEKIGKEFKRIVQRIKDFLRNLVPRT |
| | ES |
| SEQ ID NO: 34 | NKRFALLGDFFRKSKEKIGKEFKRIVQRIKDFLRNLV |
| | PR |
| SEQ ID NO: 35 | QHRYQQLGAGLKVLFKKTHRILRRLFNLAK |
| | Anti-DIG antibodies |
| SEQ ID NO: 36 | variable light chain domain VL of murine <Dig> 19-11 |
| SEQ ID NO: 37 | variable heavy chain domain VH of murine <Dig> 19-11 |
| SEQ ID NO: 38 | variable light chain domain VL of humanized <Dig> 19- |
| | 11 |
| SEQ ID NO: 39 | variable heavy chain domain VH of humanized <Dig> |
| | 19-11 |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### EXAMPLES

The following are examples of methods and compositions of the invention. It is understood that various other embodiments may be practiced, given the general description provided above.

### Experimental Procedures

Example 1: Isolation and characterization of cDNAs encoding the VH and VL domains of a murine <Dig> IgG1 kappa from mouse hybridoma clone 19-11
Example 2: Humanization of the VH and VL domains of mu<Dig> 19-11
Example 3: Composition, expression and purification of recombinant humanized <Dig> antibodies and bispecific derivatives
Example 4: Binding of recombinant humanized <Dig> antibodies, -fragments and -fusion proteins to digoxigenated compounds
Example 5: Generation of digoxigenated compounds
Example 6: Generation of defined complexes of digoxigenated compounds with <Dig> IgG
Example 7: Digoxigenated peptides and complexes with <Dig> antibodies retain functionality
Example 8: Digoxigenated antibody-complexed PYY(3-36) derived peptides have better potency than PEGYlated PYY(3-36) derived peptides in cell culture experiments
Example 9: Serum stability and serum levels of complexes of digoxigenated Cy5 or digoxigenated PYY-derived peptides with <Dig> IgG
Example 10: In vivo activity of complexes of digoxigenated PYY-derived peptides with <Dig> IgG

### Tables

Table 1: Binding affinities of the murine ‘wildtype’ DIG-IgG and recombinant <Dig> derivatives to different digoxigenated antigens
Table 2: cytotoxic potency of unmodified and digoxigenated human-derived peptides
Table 3: fluorescence of the unmodified and digoxigenated and complexed fluorophore Cy5
Table 4: Biologic activity in vitro of PYY derivatives in the cAMP assay
Table 5: PK parameters of uncomplexed and antibody-complexed Dig-fluorophore and Dig-peptide

### Example 1a

### Anti-Dig Antibodies

Antibodies that bind specifically to the cardiac glycosides digoxin, digitoxin, and digoxigenin can be generated as described e.g. in Hunter, M. M., et al, J. Immunol. 129 (1982) 1165-1172. One example of such antibody is the monoclonal antibody 26-10. The 26-10 antibody binds to the cardiac glycosides digoxin, digitoxin, and digoxigenin with high-affinity (KD=9 nM) (Schildbach, J. F., et al., J. Biol. Chem. 268 (1993) 21739-21747; Burks, E. A., et al., PNAS 94 (1997) 412-417).

By applying these methods and using a digoxin conjugated to human serum albumin for the immunization we generated the monoclonal, murine <Dig> antibody hybridoma clone 19-11.

### Example 1b

### Isolation and Characterization of cDNAs Encoding the VH and VL Domains of a Murine <Dig> IgG1 Kappa from Mouse Hybridoma Clone 19-11

A prerequisite for the design, generation, optimization and characterization of recombinant <Dig> antibodies, antibody fragments and -fusion proteins is the availability of protein and (DNA) sequence information. Therefore, from the hybridoma clone 19-11 this information for the VH and VL domains of murine <Dig> antibody was obtained. The experimental steps that needed to be performed subsequently were (i) the isolation of RNA from <Dig> producing 19-11 hybridoma cells, (ii) conversion of this RNA into cDNA, then into VH and VL harboring PCR fragments, and (iii) integration of these PCR fragments into plasmids vectors for propagation in E. coli and determination of their DNA (and deduced protein) sequences. More details of the herewith described experimental steps have been described in PCT/EP2010/004051.

RNA Preparation from 19-11 Hybridoma Cells:

RNA was prepared from 5×10e6 antibody expressing hybridoma cells (clone 19-11) applying the Rneasy-Kit (Qiagen). Briefly, the sedimented cells were washed once in PBS and sedimented and subsequently resuspended for lysis in 500 μl RLT-Puffer (+β-ME). The cells were completely lysed by passing through a Qiashredder (Qiagen) and then subjected to the matrix-mediated purification procedure (ETOH, RNeasy columns) as described in the manufacturers manual. After the last washing step, RNA was recovered from the columns in 50 ul RNase-free water. The concentration of the recovered RNA was determined by quantify A260 and A280 of 1:20 diluted samples. The integrity (quality, degree of degradation) of the isolated RNA samples was analyzed by denaturing RNA gel electrophoresis on Formamide-Agarose gels (see Maniatis Manual). Examples of these RNA gel electrophoreses, which showed discrete bands that represent the intact 18s and 28 s ribosomal RNAs. Intactness (and approx 2:1 intensity ratios) of these bands indicated a good quality of the RNA preparations. The isolated RNAs from the 19-11 hybridoma were frozen and stored at −80 C in aliquots.

### Generation of DNA Fragments Encoding 19-11 VH and VH by RACE PCR:

The cDNA for subsequent (RACE-) PCR reactions were prepared from 19-11 RNA preparations by applying the FirstChoice Kit (Ambion) reagent kit using the described reactions for a standard 5′-RLM RACE protocol. Pwo DNA polymerase was used for the PCR reaction. For that, 10 μg of 19-11 RNA or control RNA (from mouse thymus) was applied, and processed as described to integrate the 5′RACE adapter. We did not need to apply the ‘outer PCR’ reaction and directly proceeded to the ‘inner PCR’: This involved combining primer pairs consisting of the 5′RACE Inner Primer (from the kit) and either C-kappa or CH1 specific primers. The primer sequence for cKappa to amplify the VL region was 5′-TTTTTTGCGGCCGCCctaacactcattcctgttgaagctc-3′. The primer sequence for CH1 to amplify the VH region was 5′-TTTTTTGCGGCCGCGTAC ATATGCAAGGCTTACAACCACAATCC-3′. For these primer combinations, annealing temperatures of 60° C. are suitable and temperatures between 55 and 65 C/(Gradient PCR) have been applied to perform the PCR (94 C 0.5 min, 55-65 C 1 min-72 C 1 min, 35 cycles, completion by 10 min extension at 72 C). Successful specific amplification of antibody VH or VL region containing DNA fragments was reflected by occurrence of discrete 600 bp to 800 bp DNA fragments which were obtained from 19-11 RNA. These DNA fragments contain the VH and VL encoding sequences of the <Dig> hybridoma 19-11.

Cloning of the DNA Fragments Encoding 19-11 VH and VH into Plasmids and Determination of their DNA- and Protein Sequences:

The VH and VL-encoding PCR fragments were isolated by agarose gel extraction and subsequent purification by standard molecular biology techniques (Maniatis Manual). The Pwo-generated purified PCR fragments were inserted into the vector pCR bluntII topo by applying the pCR bluntII topo Kit (Invitrogen) exactly following the manufacturers instructions. The Topo-ligation reactions were transformed into E. coli Topo10—one-shot competent cells. Thereafter, E. coli clones that contained vectors with either VL- or VH containing inserts were identified as colonies on LB-Kanamycin agar plates. Plasmids were subsequently prepared from these colonies and the presence of the desired insert in the vector was confirmed by restriction digestion with EcoRI. Because the vector backbone contains EcoRI restriction recognition sites flanking each side of the insert, plasmids harboring inserts were defined by having EcoRI-releasable inserts of approx 800 bp (for VL) or 600 bp (for VH). The DNA sequence and the deduced protein sequence of the 19-11 VL and VH was determined by automated DNA sequencing on multiple clones for VH and VL. The amino acid sequence of the VL of <Dig> clone 19-11 is shown in SEQ ID NO:36 and of the VH sequence of <Dig> clone 19-11 in SEQ ID NO:37.

### Example 2

Humanization of the VH and VL Domains of mu<Dig> 19-11

The objective of humanization of antibody sequences is to generate molecules hat retain full functionality of the original antibodies of murine origin, but that harbor no (or only very few or non-relevant) sequences or structures that are recognized as ‘foreign’ by the human immune system. Different procedures are available and have been published that can address this challenge (Almagro, J. C., and Fransson, J., Frontiers in Bioscience 13 (2008) 1619-1633; Hwang, W. Y. K., and Foote, J., Methods 36 (2005) 3-10). The functionality of variable regions of antibodies is determined by secondary and tertiary (and quaternary) structures, whose formation however base on the primary sequence of VH and VL (and of adjacent and interacting entities). Because of that, the major challenge of humanization is to (fully) retain structure-defined functionality despite the need to change the primary protein sequence at some positions. Thus, knowledge about the structure of functionally important regions of antibodies (CDR regions) is very important to support humanization. To generate humanized mu<Dig> 19-11 derived variants we combined the following experimental wet-lab as well as in-silico procedures. Starting with (i) in silico-predictions of the antigen binding site of mu<Dig> 19-11 we were able to (ii) predict in-silico hu<Dig> variants with a high degree of human-likeness as well as high probability to retain full functionality. Finally (iii) we experimentally determined the (X-ray) structure of <Dig> antibody (fragments) with and without antigen to validate and improve upon our in silico model. More details of the herewith described design parameters and experimental steps have been described in PCT/EP2010/004051.

In Silico Modeling of the Antigen Binding Site of mu<Dig> 19-11:

The basis for our in-silico structure model for the mu<Dig>19-11 Fv region are the protein sequences that were deduced from the experimentally determined VH and VL mRNA sequences. A structure model of the protein encoded by these sequences was generated in silico by homology modeling of the Fv domain of the murine antibody combined with energy minimization. For that, CDRs and framework sequences to apply for the homology modeling were separately searched for homology over the PDB (Protein DataBank). For each CDR and for the frameworks, the more homolog structures were superimposed. A model was subsequently built from the different part for both the light and the heavy chains followed by a (energy) minimization of the complex. The structure model of the mu <Dig> 19-11 Fv region that resulted from our homology-modeling procedure showed that one rather particular feature of the predicted structure is a prominent cavity that appears to extend deep into the VH-VL interface. The main determinant for formation of this narrow cavity is the long CDR3 loop of VH. The interior of the cavity is lined with a methionine (deeper residue), 2 serines, 2 prolines, an a few tyrosines (flanking walls). The antigen digoxigenin that is recognized by this antibody is bound in a hapten-like manner into the deep cavity.

### Crystallization and X-Ray Structure Determination of the Binding Region of the Murine Anti-Dig Fv Region in the Presence of Antigen:

To enable further optimization of the humanized VH and VL sequences of the anti-digoxigenin antibody, we experimentally determined the structure of the parent (murine) antibody. For that, Fab fragments were generated by protease digestion of the purified IgGs, applying well known state of the art methods (papain digestion). Fab fragments were separated from remaining Fc-fragments by protein A chromatography (which removes Fc), thereafter subjected to size exclusion chromatography (Superdex200 HiLoad 120 ml 16/60 gel filtration column, GE Healthcare, Sewden) to remove protein fragments. For crystallization, purified Fabs in 20 mM His-HCl, 140 mM NaCl, pH 6.0 and Cy5 labeled Digoxigenin (DIG-3-cme-dea-Cy5=DIG-Cy5/powder) were complexed with digoxigenated fluorescent dye Cy5 (Dig-Cy5). Prior to crystal setups the protein solution was concentrated. For complex formation DIG-Cy5 was dissolved in 20 mM His-HCl, 140 mM NaCl, pH 6.0 and added to a final molar ratio of 5:1 to the concentrated protein solution. Crystals of murine Fab in complex with DIG-Cy5 were obtained using the hanging drop vapor diffusion method at 25° C. after mixing 1 μl protein solution (24 mg/ml) with 1 μl reservoir solution containing 60% (v/v) 2-methyl-1,3-propandiol (MPD)/0.1 M sodium acetate pH 4.6/5 mM CaCl₂. Crystals were flash frozen in liquid nitrogen crystals without the need of any further cryoprotection. Diffraction data of murine Fab in complex with DIG-Cy5 were collected at X06SA (SLS, Villingen, Switzerland) on Sep. 11, 2009. Data were integrated and scaled with XDS (Kabsch, J. Appl. Cryst. 21 (1993) 916-924). Crystals of the complex belong to space group P4₂2₁2 with a=b=138.01 Å, c=123.696, α=β=γ=90° and diffracted to a resolution of 2.8 Å. The structure was solved by molecular replacement using the program BALBES (see Long, F., et al., Acta Crystallogr. D Biol. Crystallogr. 64 (Pt. 1) (2008) 125-132) by generating a search model based on structures with PDB ID 3cfd, 2a6d, 2a6j (Debler, E. W., et al., Science 319 (2008) 1232-1235; Sethi, D. K., et al., Immunity 24 (2006) 429-438). In total 2 Fab molecules could be located in the asymmetric unit. The initial models were completed and refined by manual model building with the program COOT (Emsley, P., and Cowtan, K., Acta Crystallogr. D Biol. Crystallogr. 60 (Pt. 12 Pt. 1) (2004) 2126-2132) and refinement using the program PHENIX (Zwart, P. H., et al., Methods Mol. Biol. 426 (2008) 419-435). After first rounds of refinement a difference electron density for the DIG moiety of DIG-Cy5 appeared. A model for DIG was obtained from PDB ID 1lke (Korndorfer, I. P., et al., J. Mol. Biol. 330 (2003) 385-396) and refinement parameters for DIG were generated by the online tool PRODRG (Schuttelkopf, A. W., and van Aalten, D. M., Acta Crystallogr. D Biol. Crystalogr. 60 (Pt. 8) (2004) 1355-1363). The model of DIG was placed in the electron density for final refinement steps. Figures were prepared with the program PYMOL (DeLano, W. L., The PyMOL Molecular Graphics System (2008)).

The results of the experimental structure determination have been described in detail in PCT/EP2010/004051. The structure revealed that the obtained crystal form contained two independent DIG-Cy5:anti-DIG Fab complexes in the asymmetric unit and atomic models for both complexes could be build. The DIG moiety of DIG-Cy5 is well ordered in both Fab molecules in the asymmetric unit although it appears to be bound in one molecule of the asymmetric unit more tightly than in the other one. DIG is bound in a pocket located at the interface of chain L and chain H in the middle of the CDR. Atom 032 of DIG is pointing towards the bottom of the pocket and the linker with Cy5 is located outside and points into the solvent. In addition to DIG, a clear 2F_{O}-F_{C} electron density is visible for the first C atom of the linker to Cy5 (panel B in FIG. 45b). Due to the flexibility of the linker neither the remainder of the linker nor Cy5 are visible in the electron density map. This disorder indicates that the linker is not attached to the protein and long enough to allow attachment of molecules of different nature and size such as dyes, siRNA and others to DIG without influencing the recognition of DIG by the antibody. Interestingly the binding pocket is not completely hydrophobic as expected for a hydrophobic molecule as DIG but contains some positive charge potential. The binding pocket is lined by four Tyrosin residues (57, 59, 109, 110) as well as A33, W47, P61, P99 and M112 of the heavy chain. From the light chain residues Q89, S91, L94, P96 and F98 are involved in pocket formation. The possible hydrogen bonding partners N35 and Y36 of the light chain form the bottom of the pocket but are not reached by the DIG. Only one direct hydrogen bond is involved in DIG binding and is formed between O32 of DIG and Q89 of the light chain. Two more hydrogen bonds are not direct but mediated through water molecules. O12 is interacting with the carbonyl oxygen of Y109 and the side chain of S35 of the heavy chain. A fourth hydrogen bond is formed between O14 and backbone carbonyl oxygen of S91 (chain L) but again mediated by a water molecule. Comparisons of the number and the lengths of the hydrogen bonds in both molecules of the asymmetric unit indicate that in the second complex DIG is not able to fully enter the pocket. In one molecule the DIG moiety immerses relatively deep into the pocket and forms four hydrogen bonds. The second DIG is bound more loosely bound, it does not enter the pocket as deep as in the other molecule and forms only three hydrogen bonds that are weaker than in the other molecule.

Definition of mu<Dig>19-11 Humanized Variants which Retain Full Functionality:

The results of the experimental determination of the binding region at a resolution of 2.8 Å enables the characterization of the binding mode of the ligand to its antibody. It further confirms that structure is generally similar to the structure model that we predicted by in-silico analyses of the primary sequence. The availability of the in silico modeled structure as well as of experimentally determined ‘real’ structure of the variable region of the parent antibody (see PCT/EP2010/004051 for more details) is a prerequisite for detailed modeling and further improvement via protein engineering of recombinant digoxigenin binding modules. Amino acid sequences that represent desired humanized VH and VL domains were defined by applying a procedure which is based on CDR-grafting and introduction of additional mutations which modulate binding specificity and affinity. The basic principle underlying this procedure is the attribution of a ‘score value’ for each amino acid that differs from the mouse sequence among the human germlines. This score is defined by its putative influence of the amino acid change on the antigen recognition capability or on the stability of the complex. Human germline are selected based on their lower score and their relative high usage. TEPITOPE analyses (predicting T-cell epitopes) are included in this humanization procedure with the objective to have few to no t-cell epitopes in the resulting humanized molecule. The ‘human’ sequences initially defined by this procedure may need to be replaced by the (original) murine ones when the score is too high (indicating high probability of negative interference). This is most frequently required for amino acid changes in the CDR or in the surrounding region of the CDR sequences. In some instances, ‘back-mutations’ to murine residues are required not only in the CDRs but also within the framework to retain stability and functionality. The resulting hu<Dig> variant that we chose is based on the human Framework VH3_{—}11 and VL1_{—}39 combination, and has a high degree of human-likeliness. For VL, it was not necessary to integrate any backmutation in the framework of the human VK1_{—}39 and the human j element of IGKJ4-01/02 germlines. This lead to a high human character and a relatively low number of TEPITOPE alerts. The VH variant is originated from the human VH3_{—}23 germline and the human J IGHJ6-01-2. The variant J is built on the human VH3_{—}11 germline. Moreover, using our scoring methodology, we were able to introduce one human amino acid within CDRS in order to increase the human character and decrease the number of TEPITOPE alerts. The amino acid sequence of the humanized VH is shown in SEQ ID NO:38 and of the humanized VL in SEQ ID NO:39.

Generation of Digoxygenin Binding Modules with Increased Affinity:

Further optimization of the humanized VH and VL sequences of the anti-digoxigenin antibody was applied to generate modules with even higher affinity towards digoxigenin. Based upon the experimentally determined as well as in-silico calculated predicted structures (see above, based upon structure modeling without experimental structure determination), we identified three positions in which alterations might affect affinity. These were located at (Kabat positions) Ser49, Ile57 and Ala60 of the VH domain. Replacement of the amino acid VHSer49 with Ala, VHIle57 with Ala and of VHAla60 with Pro generated the respective antibody derivatives. Binding entities that are composed of this sequence could be expressed and purified with standard Protein-A and size exclusion technologies (see Example 3 ‘Composition, expression and purification of recombinant humanized <Dig> antibodies, -fragments and bispecific-fusion proteins). The resulting molecules were fully functional and displayed improved affinity towards digoxigenin compared to the humanized parent molecule. This was demonstrated by Surface-Plasmon-Resonance (BiaCore) experiments (see Example 4 ‘Binding of recombinant <Dig> antibodies, -fragments and bispecific-fusion proteins to digoxigenated antigens’ for details). The results of these experiments proved that the affinity towards digoxigenin is improved approximately 10-fold by introducing VH49, VH57 and VH60 mutations. The relevance of these positions was thereafter confirmed by inspecting the experimentally determined structure of the Dig-binding variable region.

### Example 3

### Composition, Expression and Purification of Recombinant Humanized <Dig> Antibodies

Murine and humanized <Dig> modules were combined with constant regions of human antibodies, either to form chimeric or humanized IgG's or to generate bispecific fusion proteins with other antibody sequences. The generation of humanized <Dig> IgGs that bind Dig required (i) design and definition of amino- and nucleotide sequences for such molecules, (ii) expression of these molecules in transfected cultured mammalian cells, and (iii) purification of these molecules from the supernatants of transfected cells. Also bispecific derivatives that bind Dig as well as other targets (e.g. receptor tyrosine kinases Her2 or IGF1R) were also generated as used as model systems for proof of concept studies, where e.g. the defined complexation of the peptides or fluorophores could be demonstrated in the Examples below. Additional details of the herewith described experimental steps have been described in PCT/EP2010/004051.

Design and Definition of Amino- and Nucleotide Sequences of <Dig> IgG and Bispecific Antibody Derivatives that Bind Digoxygenin as Well as HER2 or IGF1R

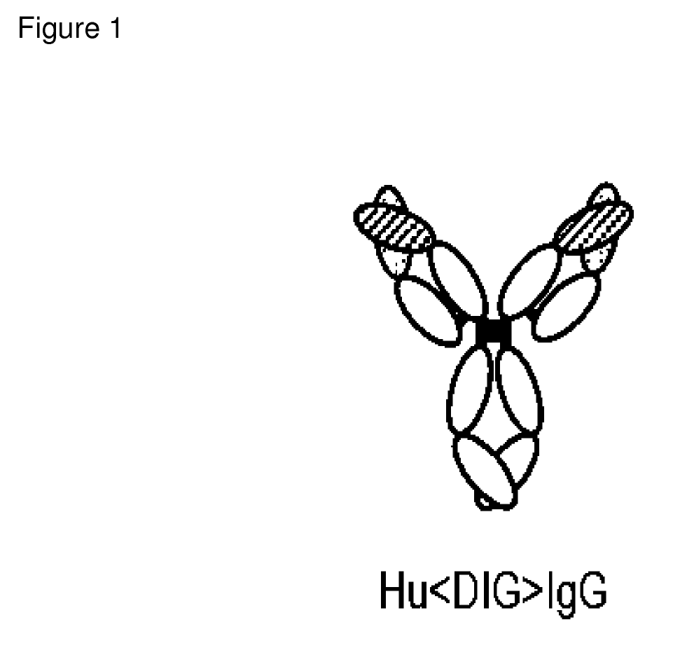

To generate a humanized IgG that harbors the binding specificity of the (original) murine mu<Dig>19-11 Fv region, we fused the above defined humanized VH sequence in frame to the N-terminus of CH1-CH2-CH3 of IgG1. Similarly, we fused the above defined humanized VL sequence in frame to the N-terminus of Ckappa. The amino acid-sequences of the resulting hu<Her2><Dig> IgG H- and L-chains have been described in PCT/EP2010/004051. A schematic representation of a humanized digoxigenin-binding IgG is provided in FIG. 1.

To generate bispecific antibody derivatives that contain the binding specificity of hu<Dig> as well as specificities to the receptor tyrosine kinase Her2 or IGF1R, we fused the <Dig> single-chain Fv module defined by humanized VH and VL sequences in frame to the C-terminus of the H-chain of a previously described <Her2> antibody (e.g. U.S. Pat. No. 5,772,997), or of a IGF1R antibody, respectively. The applied <Dig> scFv module was further stabilized by introduction of a VH44-VL100 disulfide bond which has been previously described (e.g. Reiter, Y., et al., Nature Biotechnology 14 (1996) 1239-1245). The amino acid and sequences of the resulting bispecific antibody derivatives that bind Her2 or IGF1R as well as Digoxigenin have been described in PCT/EP2010/004051.

Expression of <Dig> IgG and of Bispecific Antibody Derivatives that Bind Digoxigenin as Well as Her2 or IGF1R:

The <Dig> IgG and the bispecific antibody derivatives were expressed by transient transfection of human embryonic kidney 293-F cells using the FreeStyle™ 293 Expression System according to the manufacturer's instruction (Invitrogen, USA). For that, light and heavy chains of the corresponding bispecific antibodies were constructed in expression vectors carrying pro- and eukaryotic selection markers. These plasmids were amplified in E. coli, purified, and subsequently applied for transient transfections. Standard cell culture techniques were used for handling of the cells as described in Current Protocols in Cell Biology (2000), Bonifacino, J. S., Dasso, M., Harford, J. B., Lippincott-Schwartz, J. and Yamada, K. M. (eds.), John Wiley & Sons, Inc. The suspension FreeStyle™ 293-F cells were cultivated in FreeStyle™ 293 Expression medium at 37° C./8% CO2 and the cells were seeded in fresh medium at a density of 1-2×106 viable cells/ml on the day of transfection. The DNA-293fectin™ complexes were prepared in Opti-MEM I medium (Invitrogen, USA) using 333 μl of 293fectin™ (Invitrogen, Germany) and 250 μg of heavy and light chain plasmid DNA in a 1:1 molar ratio for a 250 ml final transfection volume. The IgG or bispecific antibody containing cell culture supernatants were clarified 7 days after transfection by centrifugation at 14000 g for 30 minutes and filtration through a sterile filter (0.22 μm). Supernatants were stored at −20° C. until purification. To determine the concentration of antibodies and derivatives in the cell culture supernatants, affinity HPLC chromatography was applied. For that, cell culture supernatants containing antibodies and derivatives that bind to Protein A were applied to an Applied Biosystems Poros A/20 column in 200 mM KH2PO4, 100 mM sodium citrate, pH 7.4 and eluted from the matrix with 200 mM NaCl, 100 mM citric acid, pH 2.5 on an UltiMate 3000 HPLC system (Dionex). The eluted protein was quantified by UV absorbance and integration of peak areas. A purified standard IgG1 antibody served as a standard.

Purification of <Dig> IgG and of Bispecific Antibody Derivatives that Bind Digoxygenin as well as Her2 or IGF1R:

7 days after transfection of the expression plasmids, the HEK293 cell supernatants were harvested. The recombinant antibody (-derivatives) contained therein were purified from the supernatant in two steps by affinity chromatography using Protein A-Sepharose™ (GE Healthcare, Sweden) and Superdex200 size exclusion chromatography. Briefly, the monospecific and bispecific antibody containing clarified culture supernatants were applied on a HiTrap ProteinA HP (5 ml) column equilibrated with PBS buffer (10 mM Na2HPO4, 1 mM KH2PO4, 137 mM NaCl and 2.7 mM KCl, pH 7.4). Unbound proteins were washed out with equilibration buffer. The bispecific antibodies were eluted with 0.1 M citrate buffer, pH 2.8, and the protein containing fractions were neutralized with 0.1 ml 1 M Tris, pH 8.5. Then, the eluted protein fractions were pooled, concentrated with an Amicon Ultra centrifugal filter device (MWCO: 30 K, Millipore) to a volume of 3 ml and loaded on a Superdex200 HiLoad 120 ml 16/60 gel filtration column (GE Healthcare, Sweden) equilibrated with 20 mM Histidin, 140 mM NaCl, pH 6.0. The protein concentration of purified antibodies and derivatives was determined by determining the optical density (OD) at 280 nm with the OD at 320 nm as the background correction, using the molar extinction coefficient calculated on the basis of the amino acid sequence according to Pace et. al., Protein Science, 1995, 4, 2411-1423. Monomeric antibody fractions were pooled, snap-frozen and stored at −80° C. Part of the samples were provided for subsequent protein analytics and characterization. The homogeneity of the DIGHu2 antibody construct and the bispecific DIG constructs were confirmed by SDS-PAGE in the presence and absence of a reducing agent (5 mM 1,4-dithiotreitol) and staining with Coomassie brilliant blue. The NuPAGE® Pre-Cast gel system (Invitrogen, USA) was used according to the manufacturer's instruction (4-20% Tris-Glycine gels). Under reducing conditions, polypeptide chains related to the IgG (and also bispecific Fv fusions) showed upon SDS-PAGE at apparent molecular sizes analogous to the calculated molecular weights. Expression levels of all constructs were analysed by Protein A. Average protein yields were between 6 and 35 mg of purified protein per liter of cell-culture supernatant in such non-optimized transient expression experiments. More details of the herewith described expression and purification steps are described in PCT/EP2010/004051.

### Example 4

### Binding of Recombinant Humanized <Dig> Antibodies, -Fragments and -Fusion Proteins to Digoxigenated Compounds

The analyses that are described below were performed to evaluate if the humanization procedure resulted in <Dig> derivatives that had retained full binding activity. For that, binding properties of the recombinant <Dig> derivatives were analyzed by surface plasmon resonance (SPR) technology using a Biacore T100 or Biacore 3000 instrument (GE Healthcare Bio-Sciences AB, Uppsala). This system is well established for the study of molecule interactions. It allows a continuous real-time monitoring of ligand/analyte bindings and thus the determination of association rate constants (ka), dissociation rate constants (kd), and equilibrium constants (KD) in various assay settings. SPR-technology is based on the measurement of the refractive index close to the surface of a gold coated biosensor chip. Changes in the refractive index indicate mass changes on the surface caused by the interaction of immobilized ligand with analyte injected in solution. If molecules bind to immobilized ligand on the surface the mass increases, in case of dissociation the mass decreases. To perform the binding studies capturing anti-human IgG antibody was immobilized on the surface of a CM5 biosensor chip using amine-coupling chemistry. Flow cells were activated with a 1:1 mixture of 0.1 M N-hydroxysuccinimide and 0.1 M 3-(N,N-dimethylamino)propyl-N-ethylcarbodiimide at a flow rate of 5 μl/min. If not described else wise, anti-human IgG antibody was injected in sodium acetate, pH 5.0 at 10 μg/ml, which resulted in a surface density of approximately 12000 RU. A reference control flow cell was treated in the same way but with vehicle buffers only instead of the capturing antibody. Surfaces were blocked with an injection of 1 M ethanolamine/HCl pH 8.5. To compare the binding of the humanized protein variants with that of the murine <Dig> IgG from the original hybridoma 19-11, capturing anti-mouse IgG antibody was immobilized on the surface of a CM5 biosensor chip in the same fashion as described above for the anti-human IgG antibody. To evaluate the functionality of the recombinant <Dig> derivatives, binding of the recombinant hu<Dig> modules, incl. (i) humanized IgG, (ii) fusion proteins harboring hu<Dig> disulfide-stabilized scFvs was assayed with digoxigenated antigens The resulting binding affinities were compared to the binding of the murine ‘wildtype’ DIG-IgG from which the recombinant humanized modules were derived.

Comparison of Hybridoma-Derived Murine <Dig> 19-11 with Humanized <Dig> IgG:

Anti-mouse IgG antibody was immobilized on the surface of a CM5 biosensor chip in the same fashion as described above. Anti-human IgG antibody was injected at 2 μg/ml, which resulted in a surface density of approximately 600 RU. The regeneration was carried out by injecting 0.85% H₃PO₄ for 60 s at 5 μl/min and then injecting 5 mM NaOH for 60 s at 5 μl/min to remove any non-covalently bound protein after each binding cycle. The samples to be analyzed were diluted in HBS-P (10 mM HEPES, pH 7.4, 150 mM NaCl, 0.005% Surfactant P20) and injected at a flow rate of 5 μl/min. The contact time (association phase) was 3 min for the antibodies at a concentration between 1 and 5 nM. In order to measure binding affinities different digoxigenated antigens were injected at increasing concentrations, that were 0.3125, 0.625, 1.25, 2.5, 5 and 10 nM for DIG-BP4. The contact time (association phase) was 3 min, the dissociation time (washing with running buffer) 5 min for each molecule at a flow rate of 30 μl/min. All interactions were performed at 25° C. (standard temperature). In case of the murine <DIG> 19_{—}11 the regeneration solution of 10 mM Glycine/HCl pH 1.5 was injected for 60 s at 30 μl/min flow to remove any non-covalently bound protein after each binding cycle. In case of the humanized <DIG> IgG the regeneration was carried out by injecting 0.85% H₃PO₄ for 60 s at 5 μl/min and then injecting 5 mM NaOH for 60 s at 5 μl/min. Signals were detected at a rate of one signal per second. The results of these analyses are shown in Table 1 and indicate that the recombinant humanized <Dig> binds digoxigenated compounds with the same functionality and high affinity as the murine parent antibody. The Kd of murine antibody towards digoxigenated protein (Dig-BP4, European Patent EP 1545623 B1) was found to be 33 pM, and that of the humanized antibody was <76 pM. Similarly, the Kd of murine antibody towards digoxigenated nucleic acids (siRNA-Dig) was found to be 269 pM, and that of the humanized antibody was 12 nM. Thus, we conclude that the functionality of the <Dig> antibody was retained in its humanized variant (The amino acid sequence of the humanized VH is shown in SEQ ID NO:38 and of the humanized VL in SEQ ID NO:39).

Comparison of Hybridoma-Derived Murine <Dig> 19-11 with Recombinant Humanized <Dig>-Single-Chain Fv-Fusion Proteins:

Anti-mouse and anti-human IgG antibodies were immobilized on the surface of a CM5 biosensor chip in the same fashion as described above. The samples to be analyzed were diluted in HBS-P and injected at a flow rate of 5 μl/min. The contact time (association phase) was 3 min for the antibodies at a concentration between 1 and 5 nM. In order to measure binding affinities different digoxigenated antigens were injected at increasing concentrations, that were 0.3125, 0.625, 1.25, 2.5, 5 and 10 nM for DIG-BP4, and between 0.018 and 120 nM for DIG-siRNA. The contact time (association phase) was 3 min, the dissociation time (washing with running buffer) 5 min for each molecule at a flow rate of 30 μl/min. All interactions were performed at 25° C. (standard temperature). The regeneration solution of 10 mM Glycine/HCl pH 1.5 was injected for 60 s at 30 μl/min flow to remove any non-covalently bound protein after each binding cycle. When RNAses were used as ligands the regeneration was carried out by injecting 0.85% H₃PO₄ for 60 s at 5 μl/min and then injecting 5 mM NaOH for 60 s at 5 μl/min. Signals were detected at a rate of one signal per second. The results of these analyses are shown in Table 1 and indicate that the recombinant humanized <Dig> scFv module that is present in the applied bispecific fusion protein (Her2-Dig,) binds digoxigenated proteins and nucleic acids with the same functionality and high affinity as the murine parent antibody. The Kd of murine antibody towards digoxigenated protein (Dig-BP4) was found to be 33 pM, and that of the humanized single-chain Fv was 68 pM. Similarly, the Kd of murine antibody towards digoxigenated nucleic acids (siRNA-Dig, see Example 11) was found to be 269 pM, and that of the humanized single-chain Fv was 35 nM. Thus, we conclude that the functionality of the wild-type antibody is also retained in the recombinant humanized <Dig> scFv module that is present in bispecific fusion proteins.

TABLE 1
Binding affinities of the murine ‘wildtype’ DIG-IgG and recombinant
<Dig> derivatives to different digoxigenated antigens

| Antibody derivative | Affinity to DIG-BP4 |
|---|---|
| murine DIG-IgG 19-11 | 33 pM |
| humanized DIG-IgG | <76 pM |
| humanized <Dig>- single- | 68 pM |
| chain Fv-fusion proteins | |

Further SPR studies were performed in which the binding affinity of the humanized <DIG>-IgG, IGF1R-DIG and the murine <DIG>M-19-11 was compared in binding to a mono-digoxigenated protein DIG-myoglobin. The binding affinities of the humanized <DIG>-IgG and of the disulfide-stabilized <DIG> scFv derivatives to DIG-Myo were comparable (˜15-25 nM) but the affinity of the murine <DIG>M-19-11 was clearly better. The higher affinities of humanized <DIG>-IgG (<76 pM, see table 1) and disulfide-stabilized <DIG> scFv to DIG-BP4 (68 pM, see table 1) are most likely due to an avidity effect of binding to DIG-BP4, because the protein DIG-BP4 carries more than one DIG molecule on its surface.

### Example 5

### Generation of Digoxigenated Compounds

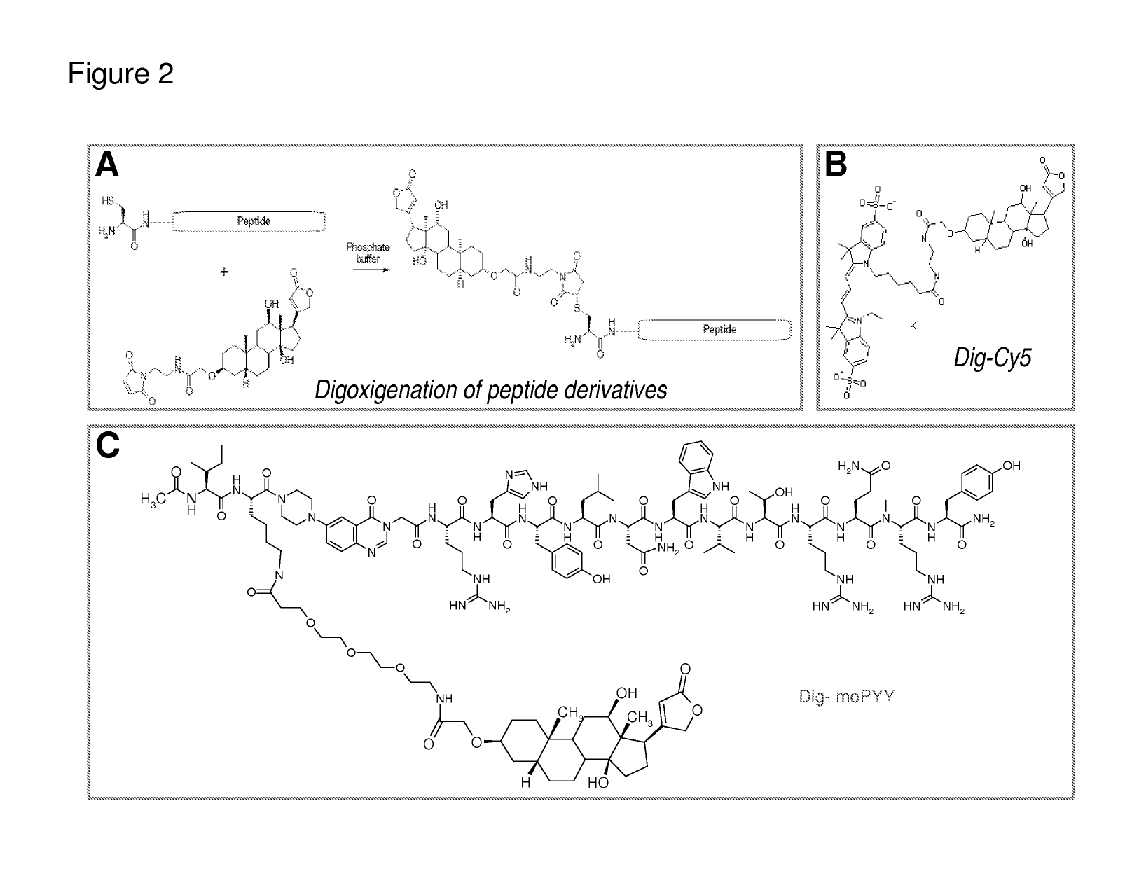

For the generation of compounds for complexation to digoxygenin-binding antibodies, it is necessary to (i) couple digoxygenin via suitable linkers to the compound and (ii) assure that the coupling occurs in a manner that allows the compound to retain its functionality. The compounds that we prepared as examples to evaluate these functionalities include a digoxigenated fluorophore (Dig-Cy5) and a set of digoxigenated peptide derivatives. The coupling procedure and reagents are schematically shown in FIG. 2A. Compositions of Dig-Cy5 and a digoxigenated PYY peptide derivative are shown in FIG. 2B and FIG. 2C, respectively. Peptides that we have used as examples to evaluate this technology are Mellittin, FALLLv1, FALLv2 and Fam5b. The latter three peptides have been identified in a screen for bioactive peptides of human origin. These peptides can be coupled to digoxygenin via addition of an amino-terminal Cystein.

The amino acid sequences of these peptides are as follows:

(SEQ ID NO: 32)

| | |
|---|---|
| Melittin: | GIGAVLKVLTTGLPALISWIKRKRQQ |

(SEQ ID NO: 33)

| | |
|---|---|
| FALLv1: | FALLGDFFRKSKEKIGKEFKRIVQRIKDFLRNLVPRTES |

(SEQ ID NO: 34)

| | |
|---|---|
| FALLv2: | NKRFALLGDFFRKSKEKIGKEFKRIVQRIKDFLRNLVPR |

(SEQ ID NO: 35)

| | |
|---|---|
| Fam5b: | QHRYQQLGAGLKVLFKKTHRILRRLFNLAK |

Another peptide derivative that we have used as examples to evaluate this technology is a PYY derivative containing unnatural amino acids. Within this text, this peptide derivative of PYY is termed moPYY (for modified PYY derivative).

The sequence of this peptide moPYY is as follows:

Ac-IK-Pqa-RHYLNWVTRQ(N-methyl)RY (SEQ ID NO: 26)

This peptide can be coupled to digoxygenin via the ε-amino group of a lysine at position 2.

Other PYY derivative peptide derivatives that can be used as examples to evaluate this technology are listed below:

(SEQ ID NO: 2)
IK-Pqa-RHYLNLVTRQRY;
(SEQ ID NO: 3)
IK-Pqa-RHYLNLVTRQ(N-methyl)RY;
(SEQ ID NO: 4)
IK-Pqa-RHYLNLVTRQ(N-methyl)R(m-)Y;
(SEQ ID NO: 5)
IK-Pqa-RHYLNLVTRQ(N-methyl)R(3-I)Y;
(SEQ ID NO: 6)
IK-Pqa-RHYLNLVTRQ(N-methyl)R(3-5 di F)Y;
(SEQ ID NO: 7)
IK-Pqa-RHYLNLVTRQ(N-methyl)R(2-6 di F)Y;
(SEQ ID NO: 8)
IK-Pqa-RHYLNLVTRQ(N-methyl)R(2-6 di Me)Y;
(SEQ ID NO: 9)
IK-Pqa-RHYLNLVTRQ(N-methyl)RF(O—CH3);
(SEQ ID NO: 10)
IK-Pqa-RHYLNLVTRQ(N-methyl)RF;
(SEQ ID NO: 11)
IK-Pqa-RHYLNLVTRQ(N-methyl)R(4-NH2)Phe;
(SEQ ID NO: 12)
IK-Pqa-RHYLNLVTRQ(N-methyl)R(4-F)Phe;
(SEQ ID NO: 13)
IK-Pqa-RHYLNLVTRQ(N-methyl)R(4-CH2OH)Phe;
(SEQ ID NO: 14)
IK-Pqa-RHYLNLVTRQ(N-methyl)R(4-CF3)Phe;
(SEQ ID NO: 15)
IK-Pqa-RHYLNLVTRQ(N-methyl)R(3-F)Phe;
(SEQ ID NO :16)
IK-Pqa-RHYLNLVTRQ(N-methyl)R(2,3.4,5,6-Penta-F)
Phe;
(SEQ ID NO: 17)
IK-Pqa-RHYLNLVTRQ(N-methyl)R(3.4-diC1)Phe;
(SEQ ID NO: 18)
IK-Pqa-RHYLNLVTRQ(N-methyl)RCha;
(SEQ ID NO: 19)
IK-Pqa-RHYLNLVTRQ(N-methyl)RW;
(SEQ ID NO: 20)
IK-Pqa-RHYLNLVTRQ(N-methyl)R(1)Nal;
(SEQ ID NO: 21)
IK-Pqa-RHYLNLVTRQ(N-methyl)R(2)Nal;
(SEQ ID NO: 22)
IK-Pqa-RHYLNLVTRQR-C-α-Me-Tyr;
(SEQ ID NO: 23)
IK-Pqa-RHYLNWVTRQ(N-methyl)RY;
(SEQ ID NO: 24)
INle-Pqa-RHYLNWVTRQ(N-methyl)RY;
(SEQ ID NO: 25)
Ac-IK-Pqa-RHYLNWVTRQ(N-methyl)R(2-6 di F)Y;
(SEQ ID NO: 27)
Pentyl-IK-Pqa-RHYLNWVTRQ(N-methyl)RY;
(SEQ ID NO: 28)
Trimetylacetyl-IK-Pqa-RHYLNWVTRQ(N-methyl)RY;
(SEQ ID NO: 29)
Cyclohexyl-IK-Pqa-RHYLNWVTRQ(N-methyl)RY;
(SEQ ID NO: 30)
Benzoyl-IK-Pqa-RHYLNWVTRQ(N-methyl)RY;
and
(SEQ ID NO: 31)
Adamtyl-IK-Pqa-RHYLNWVTRQ(N-methyl)RY.

Another compound that we have used as example to evaluate this technology is the fluorescent compound Cy5. The composition of this compound is shown in FIG. 2B. This compound can be coupled to digoxygenin via NHS-ester chemistry.

Generation of Peptides with Amino-Terminal Cystein for Digoxigenin Conjugation:

Peptide syntheses were performed according to established protocols (FastMoc 0.25 mmol) in an automated Applied Biosystems ABI 433A peptide synthesizer using Fmoc chemistry. In iterative cycles the peptide sequences were assembled by sequential coupling of the corresponding Fmoc-amino acids. In every coupling step, the N-terminal Fmoc-group was removed by treatment of the resin with 20% piperidine in N-methylpyrrolidone. Couplings were carried out employing Fmoc-protected amino acids (1 mmol) activated by HBTU/HOBt (1 mmol each) and DIPEA (2 mmol) in DMF (45-60 min vortex). After every coupling step, unreacted amino groups were capped by treatment with a mixture of Ac2O (0.5 M), DIPEA (0.125 M) and HOBt (0.015 M) in NMP (10 min vortex). Between each step, the resin was extensively washed with N-methylpyrrolidone and DMF. Incorporation of sterically hindered amino acids was accomplished in automated double couplings. For this purpose, the resin was treated twice with 1 mmol of the activated building block without a capping step in between coupling cycles. Upon completion of the target sequences, Fmoc-12-amino-4,7,10-trioxadodecanoic acid (TEG-spacer) was coupled to the FAM5B and INF7 peptides using standard amino acid coupling conditions. Subsequently, Fmoc-Cys(Trt)-OH was attached to the amino terminus of all peptide sequences (FAM5B and INF7 with spacer, Melittin, FALLv1 and FALLv2 without spacer). After final Fmoc deprotection, the peptide resin was placed into a filter frit and treated with a mixture of trifluoroacetic acid, water and triisopropylsilane (19 mL:0.5 mL:0.5 mL) for 2.5 h. The cleavage solution was filtered and the peptides were precipitated by addition of cold (0° C.) diisopropyl ether (300 mL) to furnish a colorless solid, which was repeatedly washed with diisopropyl ether. The crude product was re-dissolved in a mixture of acetic acid/water, lyophilized and subsequently purified by preparative reversed phase HPLC employing an acetonitrile/water gradient containing 0.1% TFA (Merck Cromolith prep RP-18e column, 100×25 mm).

Coupling of Peptides with Amino Terminal Cystein to Digoxigenin:

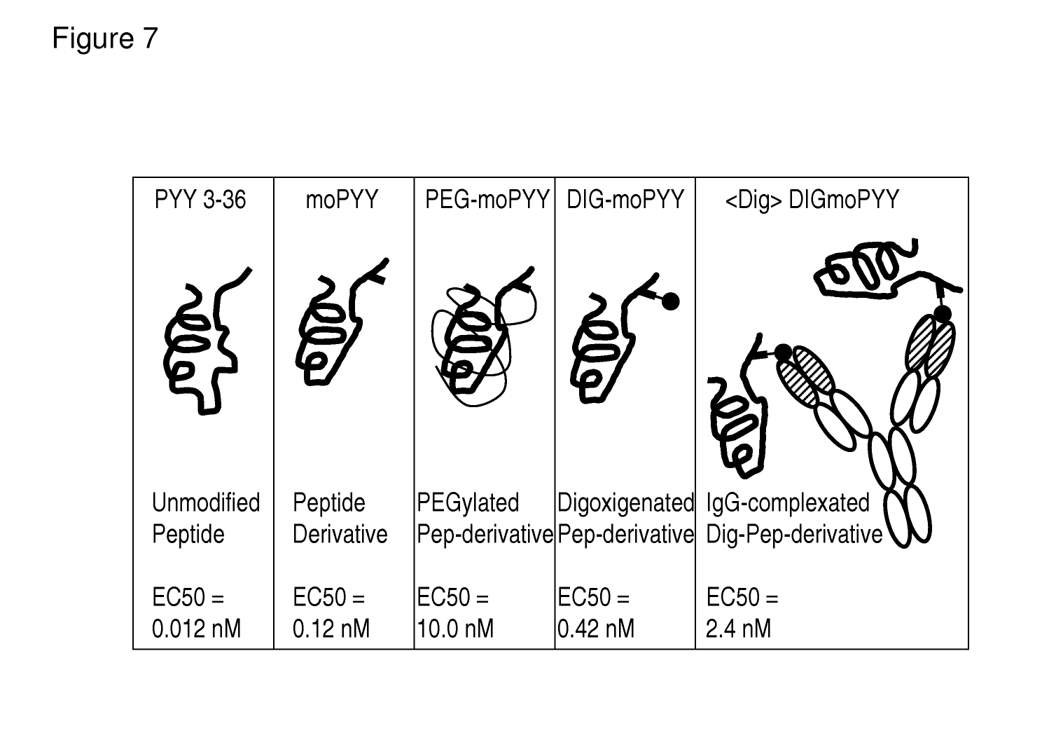

To a solution of the corresponding cysteine-modified peptide (6-20 mg) in a 0.1 M KPO₄ buffer (1 mL) was added an equimolar quantity of Digoxigenin-3-carboxy-methyl-ethylamido maleimide dissolved in 100 μL DMF. The reaction mixture was gently tumbled for 2-20 h at ambient temperature, filtered, and the target compound was isolated by preparative reversed phase HPLC employing an acetonitrile/water gradient containing 0.1% TFA (Merck Cromolith prep RP-18e column, 100×25 mm). After lyophilization the Digoxigenin-peptide conjugate was obtained as a colorless solid. The molecular weight of the peptide Melittin is 2949.64, the molecular weight of the resulting peptide-Dig conjugate is 3520.33. The molecular weight of the peptide FALLv1 is 4710.59, the molecular weight of the resulting peptide-Dig conjugate is 5384.43. The molecular weight of the peptide FALLv2 is 4791.76, the molecular weight of the resulting peptide-Dig conjugate is 5465.59. The molecular weight of the peptide Fam5b is 3634.37, the molecular weight of the resulting peptide-Dig conjugate is 5410.47. The molecular weight of the peptide INF7 is 2896.25, the molecular weight of the resulting peptide-Dig conjugate is 3466.94. Until the point of complexation to the antibody, we stored the conjugate in aliquots dissolved in H2O at −20° C. FIG. 7A represents schematically the composition of the peptide-digoxygenin conjugate.

### Generation of the Digoxigenated Form of a PYY(3-36)-Peptide Derivative:

The PYY(3-36)-peptide derivative (termed moPYY) was obtained by automated solid-phase synthesis of the resin-bound peptide sequence Ac-IK(Dde)-Pqa-R(PbOH(TrOY(tBu)LN(Trt)W(Boc)VT(tBu)R(Pbf)Q(Trt)-MeArg(Mtr)-Y(tBu)-TentaGel-RAM resin. Peptide synthesis was performed according to established protocols (FastMoc 0.25 mmol) in an automated Applied Biosystems ABI 433A peptide synthesizer using Fmoc chemistry. Employing a TentaGel RAM resin (loading: 0.18 mmol/g; Rapp Polymers, Germany), the peptide sequence was assembled in iterative cycles by sequential coupling of the corresponding Fmoc-amino acids (scale: 0.25 mmol). In every coupling step, the N-terminal Fmoc-group was removed by treatment of the resin (3×2.5 min) with 20% piperidine in N-methylpyrrolidone (NMP). Couplings were carried out employing Fmoc-protected amino acids (1 mmol) activated by HBTU/HOBt (1 mmol each) and DIPEA (2 mmol) in DMF (45-60 min vortex). At positions 2, 3, and 14, respectively, the amino acid derivatives Fmoc-Lys(ivDde)-OH, Fmoc-Pqa-OH, and Fmoc-N-Me-Arg(Mtr)-OH were incorporated into the synthesis sequence. After every coupling step, unreacted amino groups were capped by treatment with a mixture of Ac₂O (0.5 M), DIPEA (0.125 M) and HOBt (0.015 M) in NMP (10 min vortex). Between each step, the resin was extensively washed with N-methylpyrrolidone and DMF. Incorporation of sterically hindered amino acids was accomplished in automated double couplings. For this purpose, the resin was treated twice with 1 mmol of the activated building block without a capping step in between coupling cycles. After completion of the target sequence, the resin was transferred into a fitted solid-phase reactor for further manipulations.

For the removal of the ivDde group, the peptide resin (Ac-IK(Dde)-Pqa-R(Pbf)H(TrOY(tBu)LN(Trt)W(Boc)VT(tBu)R(Pbf)Q(Trt)-MeArg(Mtr)-Y(tBu)-TentaGel-RAM resin) was swelled with DMF for 30 min, and was subsequently treated with a 2% solution of hydrazine hydrate in DMF (60 mL) for 2 h. After washing the resin extensively with isopropanol and DMF, a solution of Fmoc-12-amino-4,7,10-trioxadodecanoic acid (for introducing the TEG-linker) (887 mg, 2 m mmol), HATU (760.4 mg, 2 mmol), HOAt (272.2 mg, 2 mmol) and a 2 M diisopropylethyl amine (2 mL, 4 mmol) in DMF (3 mL) was added, and the mixture was shaken for 16 h. The resin was washed with DMF and the Fmoc-group was cleaved with a mixture 40% pyridine in DMF. Subsequently, the resin was placed into a filter frit and treated with a mixture of trifluoroacetic acid, water and triisopropylsilane (19 mL:0.5 mL:0.5 mL) for 2.5 h. The cleavage solution was filtered and the peptide was precipitated by addition of cold (0° C.) diisopropyl ether (300 mL) to furnish a colorless solid, which was repeatedly washed with diisopropyl ether. The crude product was re-dissolved in a mixture of acetic acid/water and lyophilized to give the title compound as a colorless solid (337 mg, 0.137 mmol, 55%), which was used for the subsequent manipulation without further purification. For analytical characterization of the peptide derivative we applied the following conditions and received the following data: Analytical HPLC: t_{R}=9.8 min (Merck Chromolith Performance RP-18e, 100×4.6 mm, water+0.1% TFA→acetonitrile/water+0.1% TFA 80:20, 25 min); ESI-MS (positive ion mode): m/z: calcd for C₁₁₅H₁₇₃N₃₅O₂₆: 2461.9. found: 1231.7 [M+2H]²⁺, calc'd: 1231.9; 821.5 [M+3H]³⁺, calc'd: 821.6; 616.4 [M+4H]⁴⁺calc'd: 2461.9.

### Preparation of a Digoxigenated Peptide Derivative DIG-moPYY:

To a solution of peptide Ac-IK(H₂N-TEG)-Pqa-RHYLNWVTRQ(N-methyl)RY (100 mg, 40.6 μmol) in water (5 mL) was added Digoxigenin-3-carboxy-methyl-N-hydroxysuccinimide (26.6 mg, 48.8 mmol) dissolved in NMP (1 mL). Trietyhlamine (13.6 L, 97.6 μmol) was added and the mixture was tumbled for 2 h at room temperature. Subsequently, additional Digoxigenin-3-carboxy-methyl-N-hydroxysuccinimide (13.3 mg, 24.4 mmol) dissolved in NMP (0.5 mL), and triethylamine (6.8 μL, 48.8 μmol) were added and the solution was tumbled for 15 h. The crude product was purified by preparative reversed phase HPLC employing an acetonitrile/water gradient containing 0.1% TFA (Merck Cromolith prep RP-18e column, 100×25 mm) to furnish the Dig-PYY peptide (29 mg, 10.0 μmol, 25%) as a colorless solid. For analytical characterization of the peptide derivative we applied the following conditions and received the following data: Analytical HPLC: t_{R}=11.3 min (Merck Chromolith Performance RP-18e, 100×4.6 mm, water+0.1% TFA→acetonitrile/water+0.1% TFA 80:20, 25 min); ESI-MS (positive ion mode): m/z: calcd for C₁₄₀H₂₀₇N₃₅O₃₂: 2892.4. found: 964.9 [M+2H]²⁺, calc'd: 965.1. Until the point of complexation to the antibody, we stored the digoxigenated peptide as lyophilisate at 4° C. FIG. 2C shows the structure of DIG-moPYY.

### Generation of Digoxigenated Cy5:

For the generation of digoxigenated Cy5 DIG-Carboxymethyl-NHS ester (DE 3836656) was transformed with monobac ethylendiamine. Afterwards Boc was removed and the released amine was allowed to react with Cy5-NHS ester (GE Healthcare, PA15106). In order to purify DIG-Cy5 a HPLC using a RP 18 column was carried out. Eluent A was H₂O containing 0.1% TFA, eluent B was acetonitrile containing 0.1% TFA. During the elution that was run over 60 min the concentration of eluent B was increased from 0% to 100%. The molecular weight of Cy5 is 791.99 Da. The molecular weight of the resulting Cy5-Dig conjugate is 1167.55 Da. Until the point of complexation to the antibody, we stored the conjugate in aliquots in PBS at −20° C. FIG. 2B shows the structure of Cy5-digoxygenin conjugate.

### Example 6

Generation of Defined Complexes of Digoxigenated Peptides or Fluorophores with <Dig>IgG

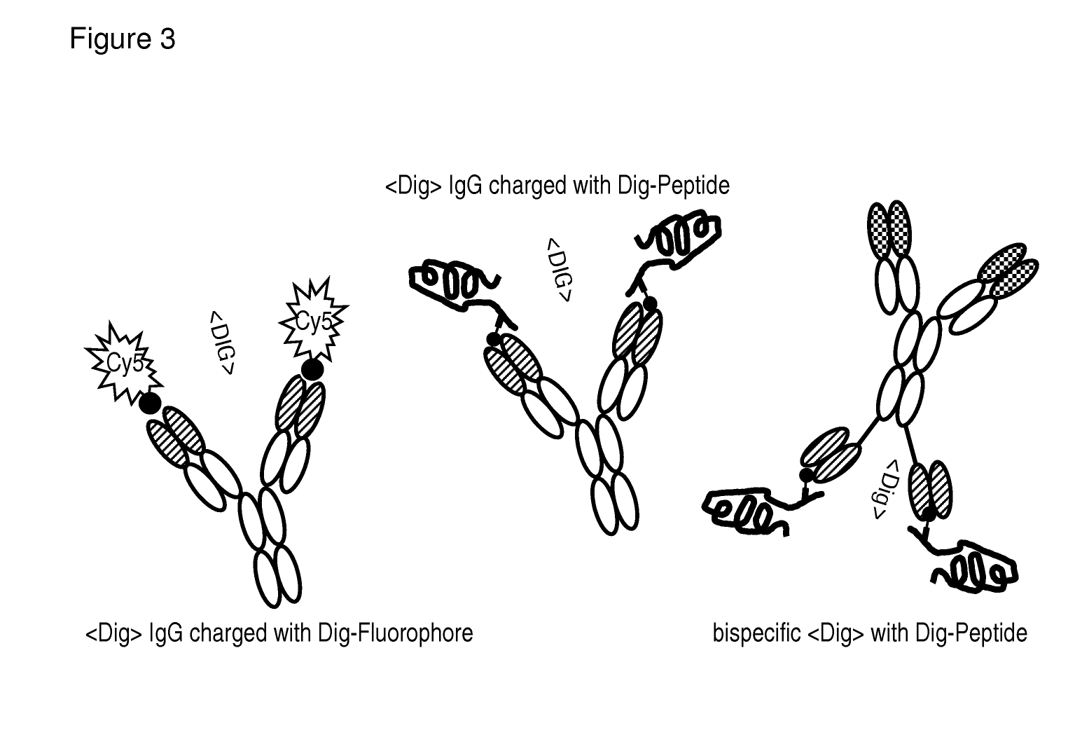

Complexes of digoxigenated peptides with digoxygenin-binding antibodies and antibody derivatives may confer benign biophysical behaviour and improved PK parameters to peptides. Furthermore, in case bispecific antibodies are applied as exemplary proof of concept complexes, such complexes are capable to target the peptides to cells which display the antigen that is recognized by the bispecific antibody variant. These complexes are composed of one humanized <Target>-<Dig> IgG which binds at its two high affinity Dig-binding sites two (one each site) digoxigenated peptides. The composition of such complexes is shown in FIG. 3. It is desired that the peptides retain good biological activity despite being digoxigenated, as well as while being complexed to the antibody. It is also desired (in case of bispecific targeting modules) that the cell surface target binding site of the bispecific antibody derivative retains its binding specificity and affinity in the presence of complexed digoxigenated peptides. One set of peptides that we have used as examples to evaluate this technology are Mellittin, FALLLv1, FALLv2 and Fam5b. The latter three peptides have been identified in a screen for bioactive peptides of human origin. The biological activity of Mellitin and the three human-derived peptides can be assessed in vitro by determining their cytotoxic effects towards human tumor cell lines. Furthermore, another peptide that we have used as an example to evaluate this technology is Peptide Tyrosine Tyrosine or Pancreatic Peptide YY short PYY(3-36) analog (WO 2007/065808). If digoxigenated via Lysine in position 2, it is called DIG-moPYY in the following text. This compound is depicted in FIG. 2C. The peptide moPYY and derivatives thereof bind to and thereby modulate the Y2 receptor (Y2R) of the NPY receptor family. PYY is secreted by the neuroendocrine cells in the ileum and colon in response to a meal. It inhibits gastric motility, increases efficiency of digestion and nutrient absorption and has been shown to reduce appetite presumably mediated by the Y2 receptor.

Because PYY plays a crucial role in energy homeostasis by balancing the food intake, this peptide may be useful to treat type II diabetes or obesity (WO 2007/065808) While moPYY is highly and specifically active in vitro it has—like many other therapeutic peptides—the disadvantage of limited stability and short serum half life in living organisms. One approach to address these issues has been site-directed PEGylation (WO 2007/065808), however PEG is known to interfere with peptide accessibility (towards receptors) and activity in many cases. The generation of antibody:Dig-peptide complexes may therefore serve as an alternative to PEGylation. For the generation of such complexes, it is necessary to (i) couple digoxygenin via suitable linkers to the peptide that allows the peptide to be exposed above the antibody surface and hence retain its activity; and (ii) generate and complexes of digoxigenated peptides with the <Dig> IgG in which the biological activity of the therapeutic peptide is retained. Another compound that we applied as examples to evaluate this technology is Dig-Cy5 (FIG. 2B). This molecule has fluorescent properties and its activity can therefore be determined by fluorescence imaging in vitro as well as in vivo.

Complexation of Digoxigenated Peptides Melittin, FALLv1 and FALLv2 with Recombinant <Target>-<Dig> Bispecific Antibodies:

For the generation of antibody complexes with digoxigenated compounds, it is necessary to (i) generate and characterize complexes of digoxigenated peptides with the digoxigenin binding antibody derivative. These complexes shall be formed in a defined manner (2 Dig-peptides bind to 1 <Dig>IgG). (ii) assure that these complexes retain activity of the compound or peptide. Recombinant <IGF1R>-<Dig> bispecific antibodies and <Her2>-<Dig> bispecific antibodies were used as protein components of the coupling reaction. The composition and purification of these molecules has been described above. For the generation of complexes of digoxigenated peptides with <IGF1R>-<Dig> and <Her2>-<Dig> bispecific antibodies, we dissolved the (Melittin, FALLv1, FALLv2) peptide-Dig conjugate in H₂O to a final concentration of 1 mg/ml. The bispecific antibody was brought to a concentration of 1 mg/ml (4.85 μM) in 20 mM Histidine, 140 mM NaCl, pH=6.0 buffer. Peptide and bispecific antibody were mixed to a 2:1 molar ratio (peptide to antibody) by pipetting up and down and incubated for 15 minutes at RT. Then, the complex was used in vitro assays without further modification. Dilutions of the complex for these assays were carried out in Opti-MEM 1 (Invitrogen Madison, Wis.). The resulting complex was defined as monomeric IgG-like molecule, carrying 2 Dig-peptides per one antibody derivative. The defined composition (and 2:1 peptide to protein ratio) of these bispecific peptide complexes was confirmed by size exclusion chromatography and charging/competition experiments. FIG. 3 provides a schematic representation of such defined antibody complexes. More details of the coupling of mellittin, FALL or Fam5B to digoxygenin-binding entities have been described in PCT/EP2010/004051.

Complexation of Digoxigenated Cy5 with <Dig> IgG and <Dig> Antibody Derivatives:

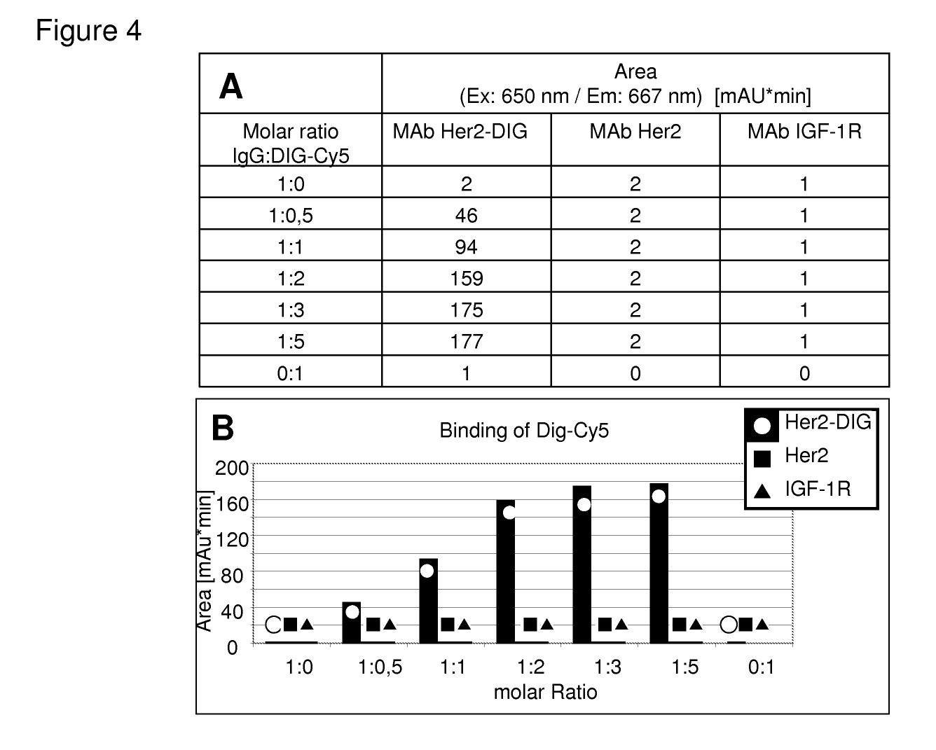

Humanized and murine <Dig> IgG or bispecific antibody derivatives were used as protein components of the coupling reaction. The composition and purification of these molecules has been described above. For the generation of complexes of digoxigenated Cy5 with digoxygenin-binding antibodies, we dissolved the Cy5-Dig conjugate in PBS to a final concentration of 0.5 mg/ml. The antibody or antibody derivative was used in a concentration of 1 mg/ml (5 μM) in a buffer composed of 20 mM Histidin and 140 mM NaCl, pH 6 (optimized results can be obtained with a concentration of the antibody or antibody derivative of at least 10 mg/ml). Digoxigenated Cy5 and antibody (-derivative) were mixed to a 2:1 molar ratio (digoxigenated Cy5 to antibody). This procedure resulted in a homogenous preparation of complexes of defined composition. FIG. 4 shows exemplarily the results of a charging experiment in which a bispecific antibody derivative containing two digoxygenin-binding sites were incubated with Dig-Cy5 in varying stoichiometric ratios. Charging of the antibody can be determined by measuring the fluorescence (650/667 nm) of the antibody-associated fluorophore on a size exclusion column. The results of these experiments demonstrate that charging occurs only if the antibody contains digoxygenin binding sites; antibodies without Dig-binding specificities (such as Her2 or IGF1R binding IgGs) do not bind Dig-Cy5. Furthermore, increased charging signals are observed for bivalent Dig-binding antibody derivatives until a Dig-Cy5 to IgG ratio of 2:1 is reached. Thereafter, charging related fluorescence signals reach a plateau. This proves that one bivalent anti-Dig IgG binds 2 molecules of Dig-Cy5. The binding complex is rather stable because it does not dissociate within the time period and under the experimental conditions that are associated with analytical size exclusion procedures

Complexation of Digoxigenated PYY(3-36)-Derived Peptides (moPYY) with Hybridoma-Derived Murine <Dig> IgG and Humanized Recombinant <Dig> IgG:

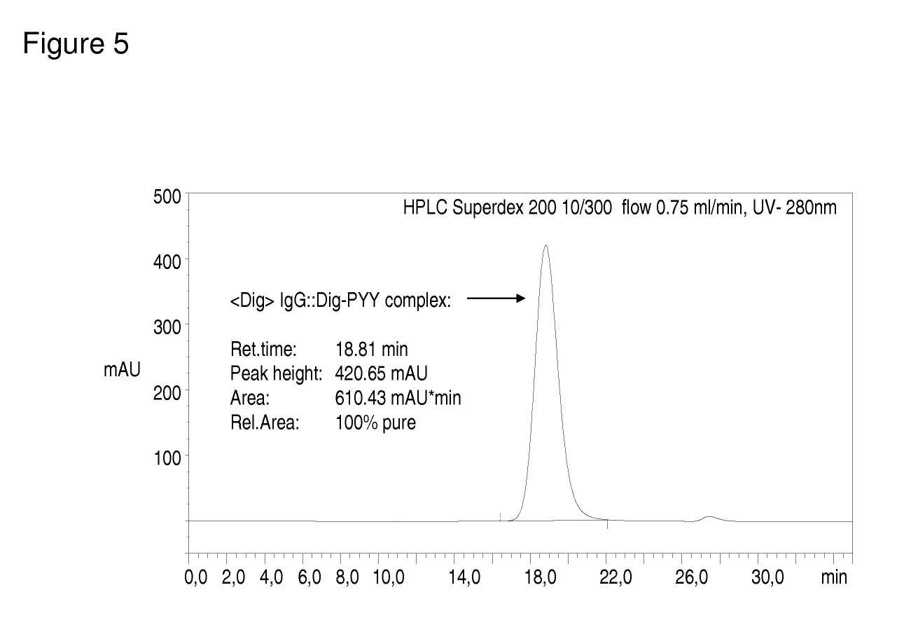

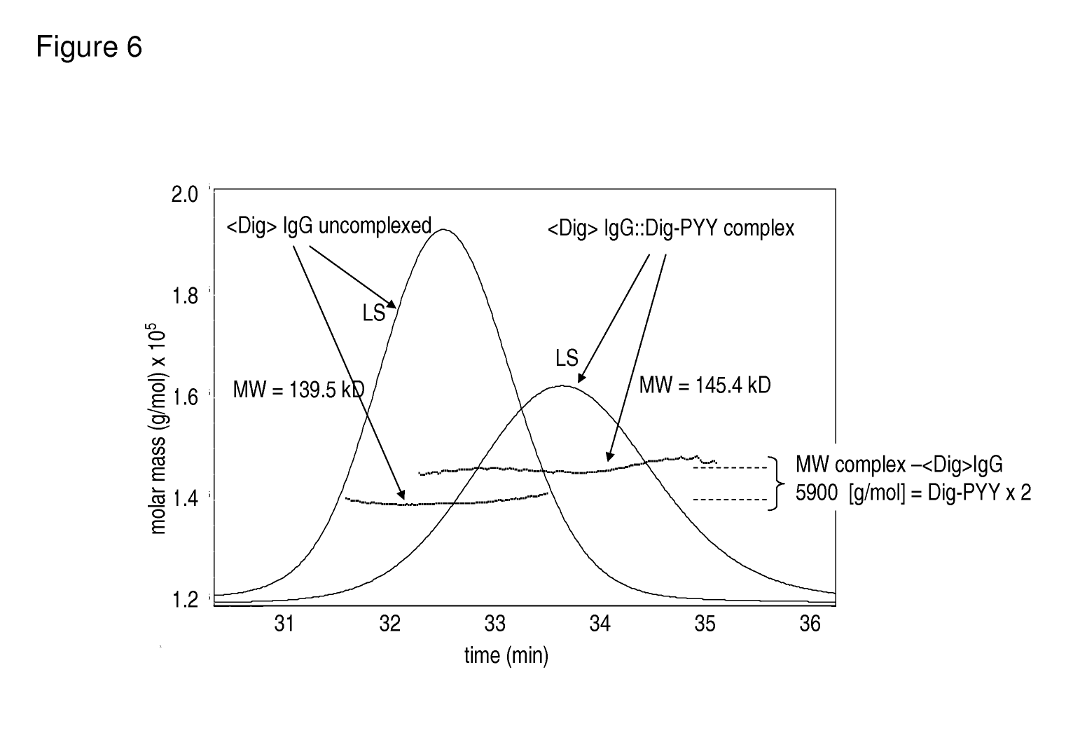

For the generation of complexes of digoxigenated peptides with the murine hybridoma-derived <Dig>IgG, the mu<IgG> (lyophilisate from 10 mM KPO4, 70 mM NaCl; pH 7.5) was dissolved in 12 ml water and dialysed against 20 mM His, 140 mM NaCl; pH 6.0 to yield 300 mg (2×10⁻⁶ mol) in 11 ml buffer (c=27.3 mg/ml). DIG-moPYY (11.57 mg, 4×10-6 mol, 2 eq.) was added in 4 portions of 2.85 mg within 1 h and incubated for another hour at room temperature. After completion of the complexation reaction, the peptide-IgG complexes were purified by size exclusion chromatography via a Superdex 200 26/60 GL column (320 ml) in 20 mM Histidin, 140 mM NaCl at pH 6.0 at a flow of 2.5 ml/min. The eluted complex was collected in 4 ml fractions, pooled and sterilized over a 0.2 μm filter to give 234 mg of the IgG/peptide complex at a concentration of 14.3 mg/ml. In a similar manner, for generation of peptide complexes of humanized <Dig> IgG, the hu<Dig> IgG was brought to a concentration of 10.6 mg/ml (9.81 mg, 6.5×10⁻⁸ mol in 0.93 ml) in 20 mM His, 140 mM NaCl, pH 6.0. 0.57 mg=1.97×10⁻⁷ mol=3.03 eq. of the digoxigenated peptide DIG-moPYY were added to the IgG solution as lyophilisate. Peptide and antibody were incubated for 1.5 hrs at room temperature. The excess of peptide was removed by size exclusion chromatography via a Superose 6 10/300 GL column in 20 mM Histidin, 140 mM NaCl at pH 6.0 at a flow of 0.5 ml/min. The eluted complex was collected in 0.5 ml fractions, pooled and sterilized over a 0.2 μm filter to give 4.7 mg of the IgG/peptide complex at a concentration of 1.86 mg/ml. The resulting peptide-IgG complex was defined as monomeric IgG-like molecule. FIG. 5 shows the size exclusion profile of the complex of DIG-moPYY peptide with the humanized and murine <Dig> IgG. The resulting complex was defined as monomeric IgG-like molecule, carrying 2 Dig-PYY derivatives per one antibody derivative. The defined composition of these peptide complexes was confirmed by size exclusion chromatography, which also indicated the absence of protein aggregates (FIG. 5). The defined composition (and 2:1 peptide to protein ratio) of these bispecific peptide complexes was further confirmed by SEC-MALS (Size exclusion chromatography-Multi Angle Light Scattering) analyses FIG. 6. For SEC-MALS analysis, 100-500 μg of the respective sample was applied to a Superdex 200 10/300 GL size exclusion column with a flowrate of 0.25-0.5 ml/min with 1×PBS pH 7.4 as mobile phase. Light scattering was detected with a Wyatt miniDawn TREOS/QELS detector, the refractive index was measured with a Wyatt Optilab rEX-detector. Resulting data was analyzed using the software ASTRA (version 5.3.4.14). The results of SEC MALLS analyses provide information about the mass, radius and size of the complex. These data were then compared with those of the corresponding uncharged antibody. The results of these experiments demonstrate that exposure of DIG-moPYY to the Dig-binding antibody results in complexes that contain two DIG-moPYY derivatives per one bivalent IgG. Thus, DIG-moPYY can be complexed with the Dig-binding antibody at defined sites (binding region) and with a defined stoichiometry. The binding complex is rather stable because it does not dissociate within the time period and under the experimental conditions that are associated with the analytical SEC-MALLS procedures. Characterization of the complex by applying surface Plasmon resonance studies provided additional evidence that the complexation reaction generated defined and completely charged molecules. Digoxygenin binding antibodies can be bound to the SPR chip which results in signal increases. Subsequent addition of DIG-moPYY results in further signal increases until all binding sites are completely occupied. At these conditions, addition of more DIG-moPYY does not increase the signal further. This indicates that the charging reaction is specific and that the signals are not caused by nonspecific stickyness of Dig-Peptides. Furthermore, the charging of the antibodies is quite stable since there is no evidence that Dig-peptides become separated from the antibody. The results of these experiments demonstrate that exposure of DIG-moPYY to the Dig-binding antibody results in a well defined composition of molecules of defined size. Thus, DIG-moPYY can be complexed with the Dig-binding antibody at defined sites (binding region) and with a defined stoichiometry. The resulting compositions appear well defined and homogenous on SEC. The binding complex is rather stable because it does not dissociate within the time period and under the experimental conditions that are associated with SEC or SPR procedures.

### Example 7

Digoxigenated Peptides and Complexes with <Dig> Antibodies Retain Functionality

One very important topic that needs to be addressed for any technology aimed at antibody-complexation of bioactive compounds is that the functionality of the compound should be retained. The antibody technology that we describe carries two modulation steps for bioactive peptides. In a first step we covalently couple digoxygenin to the bioactive peptide. In a second step, this digoxigenated peptide is complexed with the antibody derivative, which is a large protein. To retain activity of the peptide it is important to assure activity of modified peptide for both steps: activity assays need to show that (i) functionality of the peptide is retained after digoxigenation, and (ii) functionality is retained after complexation of digoxigenated peptide to the murine or humanized <Dig>.

### Comparison of the Biological Activities of Unmodified and Digoxigenated Cytotoxic Peptides and of Antibody-Complexed Cytotoxic Peptides:

To evaluate whether additions or alterations of the peptide Melittin, FALLv1 and FALLv2 by digoxygenin alters its biological activity, we performed in vitro assays. As these peptides are cytotoxic, their biological activity can easily be analyzed by monitoring the number of dead cells. To measure this number, the CytoTox-Glo assay (Promega Madison, WI) was used. Table 2 lists results of these CytoTox-Glo-assays that were performed to assess the biological activity of the Melittin, Fallv1 and Fallv2 peptides and their DIG-modified variants. For these assays, H322M cells were seeded at a density of 15.000 cells per well in 96 well plates. The cells were incubated for 24 hours at 37° C., 5% CO2 and 85% humidity in RPMI with 10% FCS, Na+ Pyrovate, L-Glutamine and NEAA mix. The peptide and it's DIG-modified variant were then added to the cells in the concentrations indicated. The cells were incubated for further 48 hours. After this period, the cells were treated with the CytoTox-Glo-assay reagent according to the manufacturers instructions. In brief, this assay detects dead cells via the presence of a protease in the medium that cleaves a fluorogenic peptide in the reagent. The luminescence of this assay therefore represents dead cells. The 96 well plates were then analyzed in a InfiniteF200 luminescence reader (Tecan Austria, Groding). The results of these assays (Table 2) show that the digoxigenated peptides retain their biological activities when compared to non-modified peptides. The IC50 value of the CytoTox-Glo assay was 3.28 μM for unmodified peptide and 3.98 μM for the digoxigenated peptide Melittin. The activities of Fallv1 and Fallv2 was similarly retained upon conjugation to digoxygenin (Table 2). Thus, digoxigenation did not interfere with the biological activity. We conclude that digoxigenation of the Melittin, FALLv1 and FALLv2 peptides does not interfere with their biological activity. Not only covalent coupling to haptens, but also complexation of peptides to large antibody molecules may influence their biological activity. Because IgG-derived molecules are large proteins (10-40 fold the size of peptides), it cannot a priori be excluded that such molecules may sterically hinder accessibility of peptide and therefore interfere with biological activity. To address this topic, we analyzed the in vitro activity of peptide-antibody complexes for the cytotoxic peptides FALLv1 and Fam5b. Again, we made use of the fact that these peptide are cytotoxic towards tumor cell lines and therefore tested their functionality in cytotoxicity and viability assays as described above. The results of these assays showed that the digoxigenated peptides retain their biological activities when complexed with digoxigenin binding antibody derivatives. Moreover, utilizing bispecific antibodies for targeting such peptides to tumor cells, we could show increased peptide-mediated cytotoxicity towards targeted cells compared to nontargeted cells (as shown in PCT/EP2010/004051).

TABLE 2
cytotoxic potency of unmodified and
digoxigenated human-derived peptides

| | IC50 unmodified | IC50 digoxigenated |
|---|---|---|
| Peptide | peptide | peptide |
| Melittin | 3.3 μM | 4.0 μM |
| FALLv2 | 9.3 μM | 7.6 μM |
| FALLv1 | 7.4 μM | 6.4 μM |

### Fluorescence Activities of Unmodified and Digoxigenated Cy5:

To evaluate whether digoxigenation of Cy5 alters its fluorescence features, we compared the excitation and emission spectra of Cy5 and compared it with the spectra of the newly generated Dig-Cy5 and with Dig-Cy5 within a complex with bispecific antibodies. Table 3 summarizes the results of these analyses: Conjugation of Cy5 to digoxygenin, as well as complexation of Dig-Cy5 to antibodies does not interfere with the fluorescence features of Cy5.

TABLE 3
fluorescence of the unmodified and digoxigenated
and complexed fluorophore Cy5

| | Excitation maximum | Emission maximum |
|---|---|---|
| molecule | (nm) | (nm) |
| Cy5 | 652 | 680 |
| DIG-Cy5 | 647 | 674 |
| <DIG> | 657 | 678 |
| DIG-Cy5 complex | | |

Comparison of the Biological Activity of Unmodified and Digoxigenated PYY(3-36)-Derived Peptides (DIG-moPYY) and Complexes with <Dig> IgG:

The desired function of PYY-derived peptides is binding to and interfering with the signaling of its cognate receptor NPY2. Signalling via the NPY2 receptor is involved in and/or regulates metabolic processes. To evaluate whether modifications of the peptide moPYY with digoxygenin to generate DIG-moPYY affect this activity, we evaluated its ability to inhibit the Forskolin stimulated cAMP accumulation in HEK293 cells expressing the NPY2 receptor (cAMP assay). In parallel, we evaluated the activity of the moPYY peptide derivatised with PEG at the same position that was used for the digoxigenation (PEG-moPYY). FIG. 7 shows the results of cAMP-assays that were performed to assess the biological activity of PYY(3-36), its Y2 receptor specific modified analog moPYY, its Dig-modified variant DIG-moPYY, of the PEGylated variant PEG-moPYY and of the antibody-complexed Dig-variant. For the cAMP agonist assay, the following materials were used: 384-well plate; Tropix cAMP-Screen Kit; cAMP ELISA System (Applied Biosystems, cat. #T1505; CS 20000); Forskolin (Calbiochem cat. #344270); cells: HEK293/hNPY2R; growth medium: Dulbecco's modified eagle medium (D-MEM, Gibco); 10% Fetal bovine serum (FBS, Gibco), heat-inactivated; 1% Penicillin/Streptomycin (Pen 10000 unit/mL: Strep 10000 mg/mL, Gibco); 500 μg/mL G418 (Geneticin, Gibco cat. #11811-031); and plating medium: DMEM/F12 w/o phenol red (Gibco); 10% FBS (Gibco, cat. #10082-147), heat-inactivated; 1% Penicillin/Streptomycin (Gibco, cat. #15140-122); 500 μg/mL G418 (Geneticin, Gibco, cat. #11811-031).

To perform the assay, on the first day, medium was discarded, and the monolayer cells were washed with 10 mL PBS per flask (T225). After decanting with PBS, 5 mL VERSENE (Gibco, cat#1504006) was used to dislodge the cells (5 min @ 37° C.). The flask was gently tapped and the cell suspension was pooled. Each flask was rinsed with 10 mL plating medium and centrifuged at 1000 rpm for 5 min. The suspension was pooled and counted. The suspension was resuspended in plating medium at a density of 2.0/105 cells/mL for HEK293/hNPY2R. 50 microliters of cells (HEK293/hNPY2R—10,000 cells/well) were transferred into the 384-well plate using Multi-drop dispenser. The plates were incubated at 37° C. overnight. On the second day, the cells were checked for 75-85% confluence. The media and reagents were allowed to come to room temperature. Before the dilutions were prepared, the stock solution of stimulating compound in dimethyl sulphoxide (DMSO, Sigma, cat#D2650) was allowed to warm up to 32° C. for 5-10 min. The dilutions were prepared in DMEM/F12 with 0.5 mM 3-Isobutyl-1-methylxanthine (IBMX, Calbiochem, cat#410957) and 0.5 mg/mL BSA. The final DMSO concentration in the stimulation medium was 1.1% with Forskolin concentration of 5 μM. The cell medium was tapped off with a gentle inversion of the cell plate on a paper towel. 50 μL of stimulation medium was placed per well (each concentration done in four replicates). The plates were incubated at room temperature for 30 min, and the cells were checked under a microscope for toxicity. After 30 min of treatment, the stimulation media was discarded and 50 μL/well of Assay Lysis Buffer (provided in the Tropix kit) was added. The plates were incubated for 45 min at 37° C. 20 μL of the lysate was transferred from stimulation plates into the pre-coated antibody plates (384-well) from the Tropix kit. 10 μL of AP conjugate and 20 μL of anti-cAMP antibody was added. The plates were incubated at room temperature while shaking for 1 hour. The plates were then washed 5 times with Wash Buffer, 70 μL per well for each wash. The plates were tapped to dry. 30 μL/well of CSPD/Saphire-II RTU substrate/enhancer solution was added and incubated for 45 min @ RT (shake). Signal for 1 sec/well in a Luminometer. (VICTOR-V) was measured. The results of these assays (FIG. 7) show that the digoxigenated peptide derivative DIG-moPYY retains most of the activity of moPYY. The IC50 value of the cAMP assay was 0.012 nM for unmodified peptide, 0.12 nM for the modified analog moPYY and 0.42 nM for the digoxigenated peptide DIG-moPYY. Thus the digoxigenation had only minor effects on the biological activity. Complexation with a large <Dig> IgG had some influence on activity of the Dig-peptide, but it still retained significant activity: the IC50 value of the cAMP assay was 2.4 nM for the peptide-antibody complex.

Also other PYY derivatives (Neuropeptide-2 receptor agonists of WO 2007/065808) showed biologic activity in vitro, as demonstrated in the cAMP assay (see WO 2007/065808) and are useful peptides for anti-<DIG>/Dig-peptide complexes. Summary of the in vitro results, EC₅₀ for are illustrated in the Table below:

TABLE 4
Biologic activity in vitro of PYY derivatives in
the cAMP assay

| Example | | Y2R |
|---|---|---|
| of | | EC50 |
| WO2007/ | | (nM) |
| 065808 | Sequence | cAMP |
| 3 | IKPEAPGEDASPEELNRYYASLRHYLNL | 0.033 |
| | VTRQRY (3-36) | |
| 4 | IK-Pqa-RHYLNLVTRQRY | 0.047 |
| 5 | IK-Pqa-RHYLNLVTRQ(N-methyl)RY | 0.42 |
| 6 | IK-Pqa-RHYLNLVTRQ(N-methyl)R(m-)Y | 1.5 |
| 7 | IK-Pqa-RHYLNLVTRQ(N-methyl)R(3-I)Y | 0.31 |
| 8 | IK-Pqa-RHYLNLVTRQ (N-methyl)R(3-5 | 0.36 |
| | di F)Y | |
| 9 | IK-Pqa-RHYLNLVTRQ(N-methyl)R(2-6 | 0.19 |
| | di F)Y | |
| 10 | IK-Pqa-RHYLNLVTRQ(N-methyl)R(2-6 | 0.67 |
| | di Me)Y | |
| 11 | IK-Pqa-RHYLNLVTRQ(N-methyl)RF(O— | 0.55 |
| | CH3) | |
| 12 | IK-Pqa-RHYLNLVTRQ(N-methyl)RF | 0.69 |
| 13 | IK-Pqa-RHYLNLVTRQ(N-methyl)R(4- | 0.31 |
| | NH2)Phe | |
| 14 | IK-Pqa-RHYLNLVTRQ(N-methyl)R(4- | 0.96 |
| | F)Phe | |
| 15 | IK-Pqa-RHYLNLVTRQ(N-methyl)R(4- | 0.45 |
| | CH2OH)Phe | |
| 16 | IK-Pqa-RHYLNLVTRQ(N-methyl)R(4- | 3.55 |
| | CF3)Phe | |
| 17 | IK-Pqa-RHYLNLVTRQ(N-methyl)R(3- | 0.75 |
| | F)Phe | |
| 18 | IK-P qa-RHYLNLVTRQ (N- | 2.5 |
| | methyl)R(2,3.4,5,6-Penta-F)Phe | |
| 19 | IK-Pqa-RHYLNLVTRQ(N-methyl)R(3.4- | 1.47 |
| | diC1)Phe | |
| 20 | IK-Pqa-RHYLNLVTRQ(N-methyl)RCha | 0.5 |
| 21 | IK-Pqa-RHYLNLVTRQ(N-methyl)RW | 1.06 |
| 22 | IK-Pqa-RHYLNLVTRQ(N-methyl)R(1)Nal | 1.14 |
| 23 | IK-Pqa-RHYLNLVTRQ(N-methyl)R(2)Nal | 2.4 |
| 24 | IK-Pqa-RHYLNLVTRQR-C-α-Me-Tyr | 1.35 |
| 25 | IK-Pqa-RHYLNWVTRQ(N-methyl)RY | 0.25 |
| 26 | INle-Pqa-RHYLNWVTRQ(N-methyl)RY | 0.108 |
| 27 | Ac-IK-Pqa-RHYLNWVTRQ(N-methyl)R(2- | 0.07 |
| | 6 di F)Y | |
| 28 | Ac-IK-Pqa-RHYLNWVTRQ(N-methyl)RY | 0.18 |
| 29 | Pentyl-IK-Pqa-RHYLNWVTRQ(N- | 0.51 |
| | methyl)RY | |
| 30 | Trimetylacetyl-IK-Pqa-RHYLNWVTRQ | 0.26 |
| | (N-methyl)RY | |
| 31 | Cyclohexyl-IK-Pqa-RHYLNWVTRQ(N- | 1.37 |
| | methyl)RY | |
| 32 | Benzoyl-IK-Pqa-RHYLNWVTRQ(N- | 0.66 |
| | methyl)RY | |
| 33 | Adamtyl-IK-Pqa-RHYLNWVTRQ(N- | 2.9 |
| | methyl)RY | |

### Example 8

Digoxigenated Antibody-Complexed moPYY Peptides have Better Potency than PEGYlated moPYY Peptides in Cell Culture Experiments.

Covalent coupling of PEG to peptides frequently interferes with the functionality of peptides and hence reduce their activity. For example, PEG chains that are frequently longer than peptides to which they are attached may ‘wrap around’ the peptides and thereby cover accessibility of essential regions. It is possible that not only covalent coupling to haptens, but also complexation of peptides to large antibody molecules may influence biological activity. It appears unlikely that IgG's can ‘wrap around’ Peptides like PEG chains and thereby cover accessibility of essential regions. However, since IgGs are large proteins (10-40 fold the size of peptides), it cannot a priori be excluded that such molecules may sterically hinder accessibility of peptide and therefore interfere with biological activity. To address this topic, we compared the in vitro activity of peptide-IgG complexes vs PEG-Peptide for the PYY-derived peptide in cAMP assays that address peptide interactions with its cognate receptor (see above for details for the cAMP assay).

The results of these assays (FIG. 7) show that the digoxigenated peptide retains activity better than its PEGylated counterpart. The IC50 value of the cAMP assay was 0.42 nM for the digoxigenated peptide DIG-moPYY. In contrast, PEGylation at the same position as in PEG-moPYY resulted in a molecule with greatly (>20 fold) decreased potency (IC50=10 nM). This shows that digoxigenation of the PYY(3-36) analog peptide has less impact on its biological activity compared to PEGylation at the same position. Furthermore, the improved potency of Dig-peptides vs PEG-peptides is still seen upon complexation with <Dig> antibody: The IC50 value of the cAMP assay was 2.4 nM for the peptide-antibody complex compared to 10 nM for the PEGylated peptide. Thus, the biological activity in vitro was four fold better for the Dig-peptide-antibody complex compared to PEG-peptide in vitro.

### Example 9

Serum Stability and Serum Levels of Complexes of Digoxigenated Cy5 or Digoxigenated moPYY Peptides with <Dig> IgG

The objective of our peptide modification technology is to improve the therapeutic applicability of peptides. Major bottlenecks for therapeutic application of peptides are currently limited stability in vivo and/or short serum half life and fast clearance. To evaluate if complexation of hapten-labeled peptides with antibodies may overcome these issues, we determined the PK parameters of antibody complexes of fluorophores or peptides in vivo and compared them with the PK of unmodified compounds. To do that we needed to (i) charge the digoxygenin-binding IgG with digoxigenated fluorophore Dig-Cy5 or digoxigenated Dig-PYY peptide derivative; (ii) apply uncomplexed and complexed compounds to animals and (iii) analyze the serum concentrations of the compounds over time in these animals.

Preparation of <Dig-IgG> Complexes with Dig-Cy5 and DIG-moPYY:

To generate Dig-Cy5 complexes, 886.1 nmol of lyophilized DIG-Cy5 were added to 446.4 nmol anti-DIG-Antibody in 20 mM Histidin/140 mM NaCl pH 6.0 in 4 portions within 1 h at RT, slowly shaking. After the addition of the last portion the sample was incubated for a total of 2 h. In case of the DIG-PYY complexes, 691.7 nmol of lyophilized DIG-PYY were added to 364 nmol anti-DIG-Antibody in 20 mM Histidin/140 mM NaCl pH 6.0 and treated equally as the DIG-Cy5 complexes. The samples were applied to a Superdex 200 HiLoad 16/60 prep grade size exclusion column with 20 mM Histidin/140 mM NaCl pH6.0 as mobile phase. Fractions containing the complex were pooled and concentrated to 19.4 mg/ml (DIG-Cy5 complex) and 19.9 mg/ml (DIG-PYY complex) with a centrifugal filtration device (Vivaspin 20, 30 kDa MWCO, GE Healthcare). The protein concentration of the DIG-Cy5 containing sample was determined by the formula 4A₂₈₀−(A₆₄₉×CF))×dilution factor)/ε. CF is the correction factor A₂₈₀ₙₘ/A₆₄₉ₙₘ which was determined as 0.008. Loading of the antibody with DIG-Cy5 was calculated as 1.2 moles of DIG-Cy5 per mole antibody with the formula: (A₆₄₉ₙₘ/(ε_{Cy5}×protein concentration M))×dilution factor. The loading of the DIG-PYY complexes was determined by SEC-MALLS, which resulted in a DIG-PYY:antibody ratio of 1:1. All samples were filtered with a PVDF syringe filter (0.22 μm pore size) under sterile conditions.

Determination of the Serum Concentrations of Complexed and Uncomplexed Dig-Cy5 In Vivo at Different Time Points after i.v. Application:

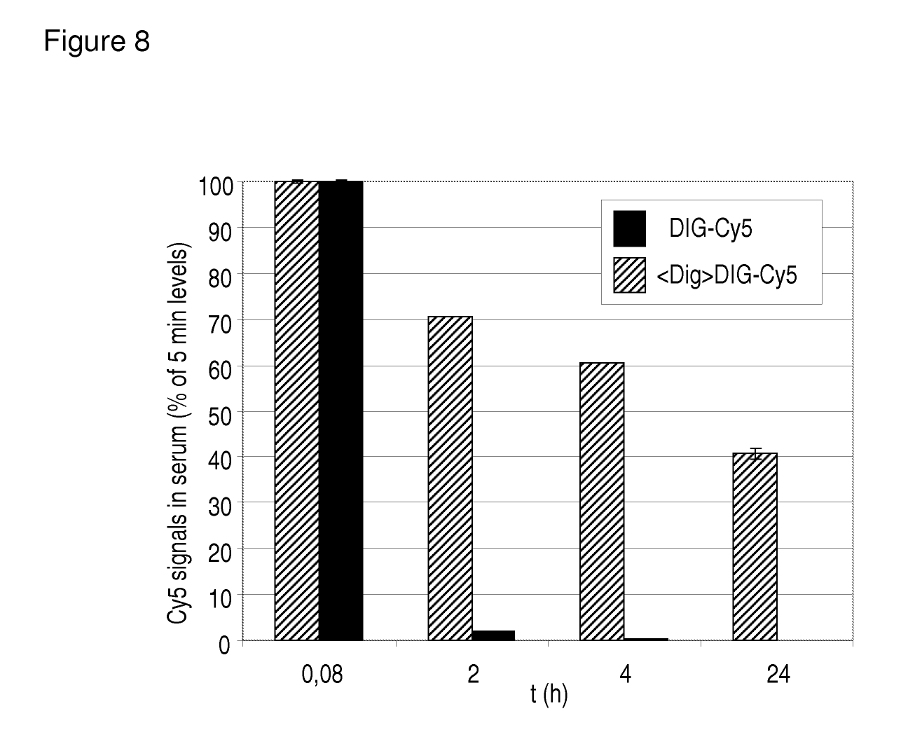

To analyze the influence on PK parameters of antibody-complexation of a small fluorescent substrate, 32.1 nmol of DIG-Cy5, or of the corresponding antibody complexated compound in 20 mM Histidin/140 mM NaCl pH 6.0 were applied to 2 female mice (strain NRMI) for each substance. The mice had a weight of 24 g, 25 g for the antibody complex and 24 g and 25 g for uncomplexed DIG-Cy5. About 0.1 ml blood samples were collected after the following time points: 0.08 h, 2 h and 24 h for Mouse 1 and 0.08 h, 4 h 24 h for Mouse 2. Serum samples of at least 40 μl were obtained after 1 h at RT by centrifugation (9300×g, 3 min, 4° C.). Serum samples were stored at −80° C. To determine the amount of compound in the serum at the given time points, we made use of the fluorescent properties of Dig-Cy5: Cy5 related fluorescence in serum samples was measured in 120 μl quartz cuvettes at room temperature using a Cary Eclipse Fluorescence Spectrophotometer (Varian). Exitation wavelength was 649 nm, Emission was measured at 670 nm. Serum samples were diluted in 1×PBS to reach an appropriate range of Emission intensity. Blood serum of an untreated mouse in the same dilution in 1×PBS as the respective sample was used as a blank probe. FIG. 8 and Table 4 shows the results of these analyses, represented as relative (%) levels of Cy5-mediated fluorescence normalized to the (peak) serum levels 5 min after injection. As expected for a compound of rather small molecular weight, uncomplexed Dig-Cy5 disappears rapidly from the serum. 2 hrs after injection, less than 5% of the fluorescence that was applied and detectable after 5 minutes in the serum was still detectable. At later time points, 4 hrs and 24 hrs after injection, Cy5-mediated signals were not detectable. This indicates rapid clearance of the compound from the circulation. In contrast to uncomplexed compound, antibody-complexed compound was detectable at higher levels and at later time points. 2 hrs after injection, still approx 70% of the fluorescence that was applied (5 min levels set to 100%) was detectable in the serum. Significant Cy5-mediated fluorescence levels were also detectable at later time points with approx 60% of the 5 min values detectable at 4 hours (hrs) and still approx 40% detectable 24 hrs after injection. This indicates that antibody complexation significantly increases the serum half life of a small compound.

TABLE 5
PK parameters of uncomplexed and antibody-
complexed Dig-fluorophore and Dig-peptide

| | | <Dig> | | <Dig> |
|---|---|---|---|---|
| | Dig-Cy5 | Dig-Cy5 | DIGmoPYY | DIGmoPYY |
| Descrip- | Digoxi- | IgG- | Digoxi- | IgG- |
| tion | genated | complexated | genated | complexated |
| | Fluorophore | Dig- | Pep- | Dig-Pep- |
| | | Fluorophore | derivative | derivative |
| Dose | 0.1 μMol/kg | 0.1 μMol/kg | 0.1 μMol/kg | 0.1 μMol/kg |
| PK Assay | Cy5- | Cy5- | Dig-Pep | Dig-Pep |
| | fluorescence | fluorescence | Western Blot | Western Blot |
| T = 5 min | 100% | 100% | (+/−) very | +++ (strong |
| (100%) | | | weak | signal) |
| T = 2 hr | <5% | 70% | − | ++ |
| T = 4 hr | <1% | 60% | − | ++ |
| T = 24 hr | not detectable | 40% | − | + |

Determination of the Serum Concentrations of Complexed and Uncomplexed Dig-Peptides In Vivo at Different Time Points after i.v. Application:

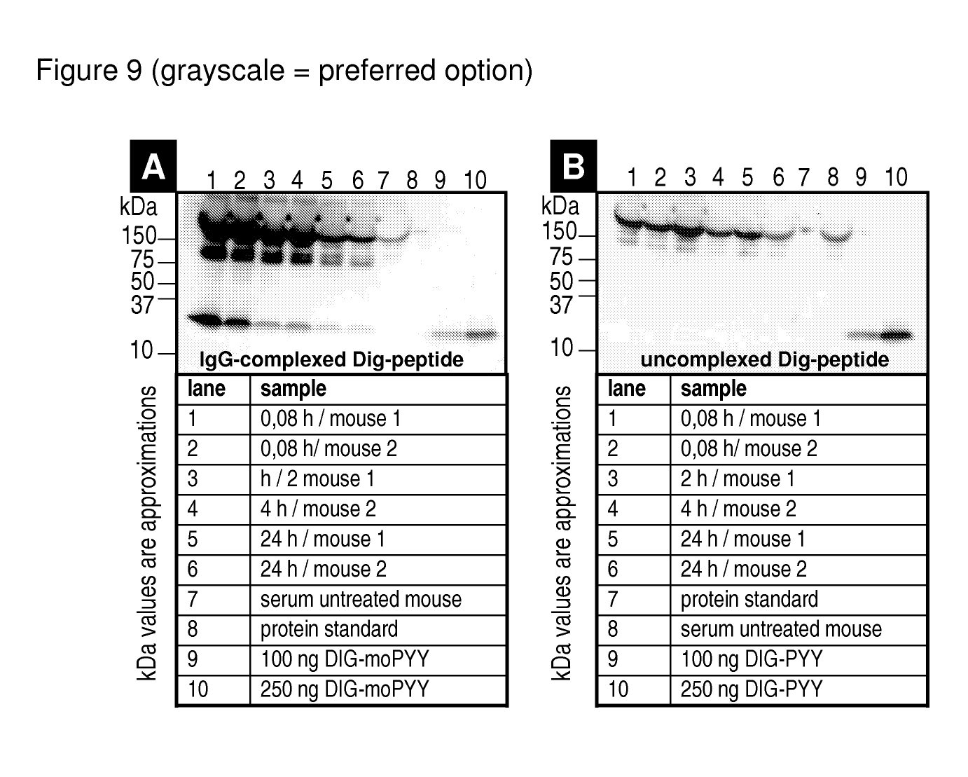

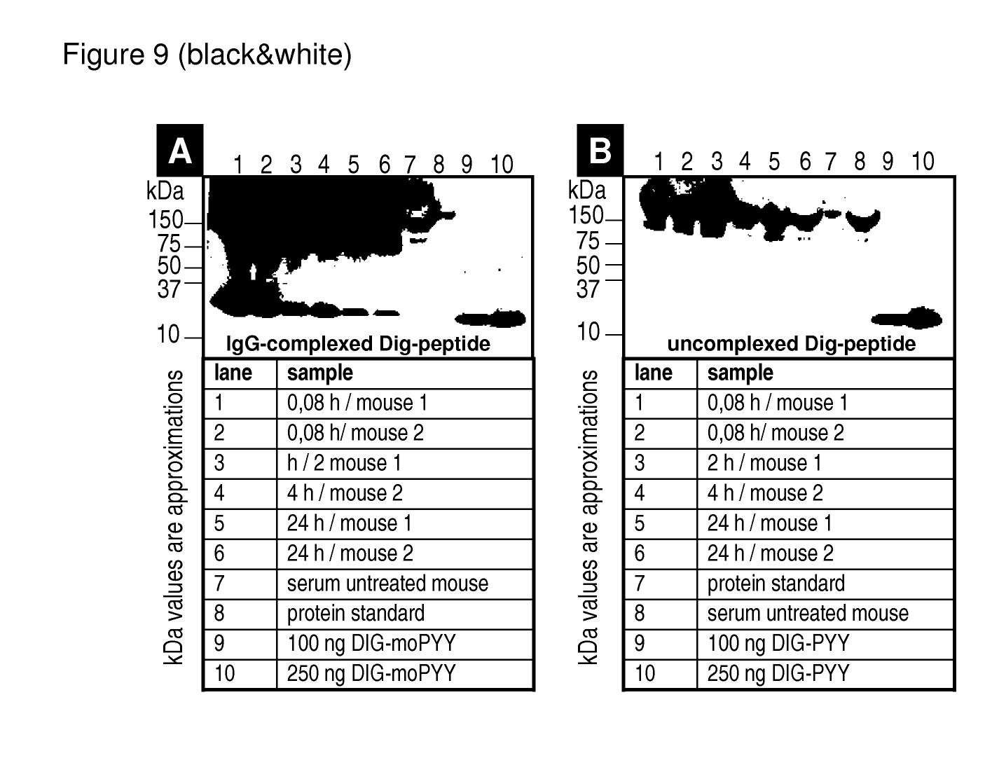

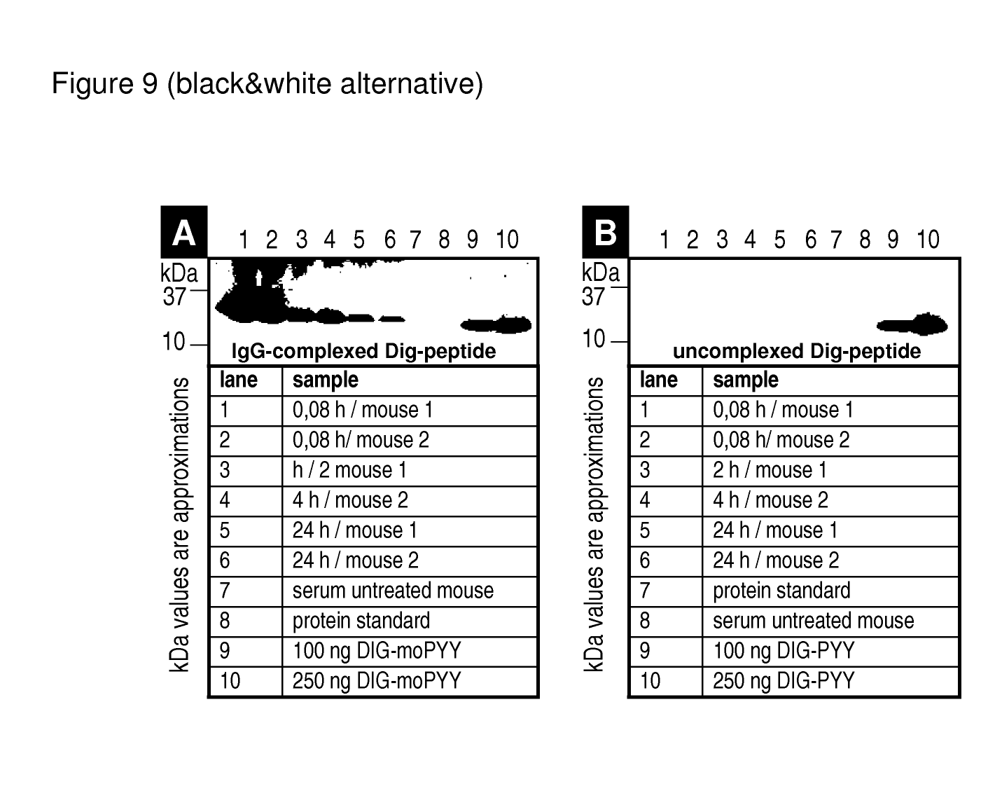

To analyze the influence on PK parameters of antibody-complexation of the digoxigenated peptide, 32.1 nmol of the peptide DIG-moPYY, or of the corresponding antibody complexated peptide in 20 mM Histidin/140 mM NaCl pH 6.0 were applied to 2 female mice (strain NRMI) for each substance. The mice had a weight of 23 g and 25 g for <DIG>-DIG-moPYY and 28 g and 26 g for DIG-moPYY. About 0.1 ml blood samples were collected after the following time points: 0.08 h, 2 h and 24 h for Mouse 1 and 0.08 h, 4 h 24 h for Mouse 2. Serum samples of at least 40 μl were obtained after 1 h at RT by centrifugation (9300×g, 3 min, 4° C.). Serum samples were stored at −80° C. The determination of the amount of digoxigenated peptide in the serum at the given time points proved to be more challenging than that of Dig-Cy5. The reason for that was that we had no direct means to detect the peptide in serum samples. Therefore, we devised a Western-Blot related assay to detect digoxigenated peptide in serum. In a first step, the serum samples were separated on reducing SDS-PAGE. Because sample preparation for that included exposure of the serum to high concentrations of SDS and reducing agents, Dig-peptides can become released from the (completely denatured/unfolded)<Dig> IgG. To mediate the release of peptide from the antibody complex and separate them by SDS-PAGE, 2 μl of each serum sample was diluted in 18 μl 20 mM Histidin/140 mM NaCl pH 6.0, mixed with 6.7 μl of 4×LDS sample buffer and 3 μl of 10× sample reducing agent (NuPAGE, Invitrogen) for 5 min at 95° C. As a control, 2 μl of serum of an untreated mouse of the same strain was used. Samples were applied to a 4-12% Bis-Tris Gel (NuPAGE, Invitrogen) which was run at 200 V/120 mA for 20 minutes using 1×MES (Invitrogen) as a running buffer. Subsequently, separated proteins and peptide were blotted onto a PVDF membrane (0.22 μm pore size, Invitrogen) using the XCell Sure Lock® Mini-Cell system (Invitrogen) for 40 min at 25 V/130 mA. Membranes were blocked in 1% skim milk in 1×PBS+1% Tween20 (PBST) for 1 h at RT. Digoxigenated peptides were subsequently detected on the membrane with anti-digoxygenin antibodies. For that, anti-Digoxigenin Antibody MAK<DIG>M-19-11-IgG(SP/Q) was applied to the membranes in a concentration of 13 μg/ml in 10 ml of 1% skim milk/PBST for 2 h at RT. Membranes were washed for 3×5 min in 1×PBST. Anti-Mouse IgG Fab-fragments coupled to POD from the LumiLight^{PLUS} Western Blotting Kit (Roche) was applied in a 1:25 dilution in 10 ml of 1% skim milk/PBST for 1 h at RT. Membranes were washed 3×5 min with 1×PBST. Detection was carried out by incubating the membranes in 4 ml LumiLight Western Blotting substrate for 5 min at RT. Chemiluminescence was detected with the LumiImager F 1 (Roche) with an exposure time of 20 min. The results of our analyses are shown in FIG. 9 and Table 4. Due to the high protein concentrations in serum and due to the rather small size of the Dig-peptide an exact quantification of the Western-Blot derived signals is not warranted. Furthermore, Western Blot derived techniques deliver qualitative (presence vs absence of bands) rather than quantitative data. Nevertheless, despite of these technical limitations, we were able to demonstrate the presence of Dig-peptides in murine serum at different time points. Mice that had received antibody complexed peptides (FIG. 9A) showed strong signals at the earliest time point (5 min after administration). These signals were clearly assignable to peptides as shown by the size and location on the blot of the control peptides. In these serum samples, additional signals of higher mass were also visible. These may represent peptides which are present in the serum and show abnormal electrophoretic behaviour in our assay. In sera of mice that were treated with antibody-complexed peptide, peptide-associated signals were strongest at the early time points and decreased over time. Nevertheless, peptides were still detectable with good signals at all time points and even 24 hrs after administration. In contrast, in mice that received uncomplexed peptides, barely any signal associatable to the small released peptide was detectable even at the earliest time point. FIG. 9B shows that under normal exposure conditions, free peptide is barely visible on the blot. Contrast enhancement and longer exposure times of the blot is capable to demonstrate the presence of some peptide 5 min after administration, however only in trace amounts. At later time points, no defined peptide band are detectable even with contrast enhancement. Furthermore, the additional signals of higher mass were also much weaker in mice that received uncomplexed peptides. We conclude from these experiments that uncomplexed peptides have a very short half life in the serum of mice. In contrast, mice that received the same peptides but in antibody complexed form, show presence of these peptides in the serum for a greatly increased period of time. Even 24 hrs after injection, peptides can be clearly identified in the serum of these mice. Thus, antibody complexation improves not only the pharmacokinetic of small fluorescent compounds (see FIG. 8) but also that of digoxigenated peptides.

### Example 10

In Vivo Activity of Complexes of Digoxigenated PYY-Derived Peptides with <Dig> IgG

The objective of the described peptide modification technology is to improve the therapeutic applicability of peptides. Major bottlenecks for therapeutic application of peptides are currently limited stability in vivo and/or short serum half life and fast clearance. Short serum half life and fast clearance in turn frequently limits the therapeutic efficacy of therapeutic peptides. Since complexation of hapten-labeled peptides with antibodies increases the serum half life of small compounds including peptides (see above), we reasoned that this might lead to improved therapeutic efficacy of antibody complexed peptides in comparison to uncomplexed peptides. To address this topic, we determined the in vivo biological activity of peptide-antibody complexes and compared them with that of uncomplexed peptides.

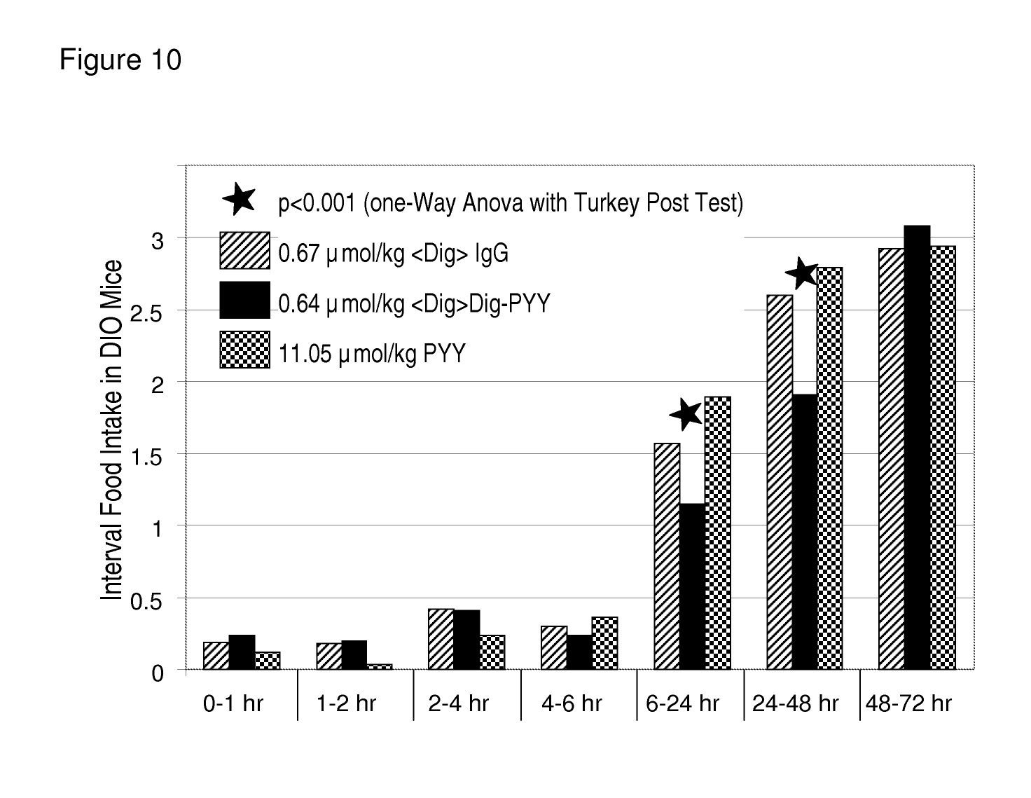

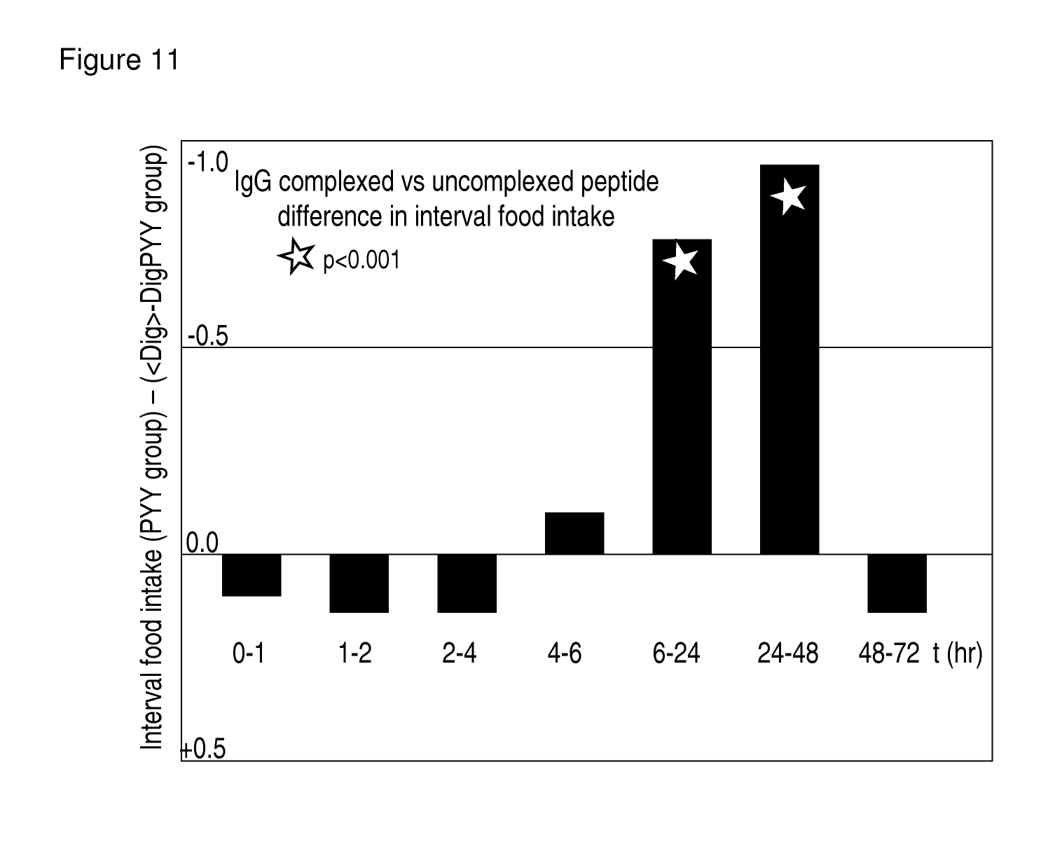

To determine the effect of NPY2-receptor agonists on food intake in these experiments we applied uncomplexed PYY-derived peptides and antibody complexed peptides to animals (adult male C57Bl/6 mice) in a DIO (diet-induced-obesity) model. The experiments were conducted on adult male C57Bl/6 mice obtained from Jackson laboratories. The mice were placed on a high fat diet (HFD; 60% of dietary kcal as fat, BioServe F3282) for over 20 weeks to induce obesity. The diet-induced obese (DIO) mice were sorted by body weight and 24 h food intake, and housed individually in standard caging at 22° C. in a reversed 12-h light/12-h dark cycle, and were acclimated to these conditions for at least 6 days before start of the experiment. Food (HFD) and water were provided ad libitum throughout the study. Ab-PYY₃₋₃₇ fusion proteins and the vehicle controls were injected at the beginning of the dark cycle and food intake measured at various time intervals up to 96 h post-dosing (N=6-8 mice/group). Uncomplexed peptide moPYY was applied at concentration of 11.05 μMol/kg. Antibody-complexed DIG-moPYY was applied at a concentration of 0.62 μMol/kg. Thus, the injected molar concentration of the antibody complexed peptide was more than 17-fold lower than that of the uncomplexed peptide. The Peptide Tyrosine Tyrosine or Pancreatic Peptide YY short PYY(3-36) analog moPYY binds to and thereby modulates the Y2 receptor (Y2R) of the NPY receptor family. PYY is secreted by the neuroendocrine cells in the ileum and colon in response to a meal. It inhibits gastric motility, increases efficiency of digestion and nutrient absorption and has been shown to reduce appetite presumably mediated by the Y2 receptor. Because PYY plays a crucial role in energy homeostasis by balancing the food intake, this peptide and derivatives thereof such as moPYY or PEG-moPYY or DIG-moPYY may be useful to treat type II diabetes or obesity. Because the peptide has been shown to reduce appetite presumably mediated by the Y2 receptor, its in vivo activity can be assessed by determining the food uptake in the DIO model. Peptide-mediated activity is thereby reflected by reduced food intake. Decreases in food intake are indicative for therapeutic efficacy, no changes in food intake or gains in intake would correspond to low efficacy or inactivity. The results of these studies are shown in FIG. 10 and FIG. 11, where differences in food intake of untreated animals are compared with those that are treated with uncomplexed peptide and those that received antibody complexed peptide. The presented data demonstrate that the application of the uncomplexed peptide has barely any effect on food intake in this animal model. In contrast, application of antibody complexed peptide (even at an almost 20-fold reduced dose) strikingly reduced the food-intake of the treated animals for a duration of hours to days. This demonstrates that stable complexation of therapeutic peptides with antibodies can significantly improve their therapeutic efficacy.

1. A pharmaceutical composition comprising a complex of:
a) a monospecific antibody that binds to digoxigenin, and
b) digoxigenin wherein the digoxigenin is conjugated to a peptide consisting of 5 to 60 amino acids.

2. The pharmaceutical composition of claim 1, wherein the peptide comprises 10 to 50 amino acids.

3. The pharmaceutical composition of claim 1, wherein the antibody of a) is a monoclonal antibody.

4. The pharmaceutical composition of claim 3, wherein the antibody of a) comprises a heavy chain variable domain of SEQ ID NO:37 and a light chain variable domain of SEQ ID NO:36.

5. The pharmaceutical composition of claim 3, wherein the antibody of a) is a humanized or human antibody.

6. The pharmaceutical composition of claim 5, wherein the antibody of a) comprises a heavy chain variable domain of SEQ ID NO:39 and a light chain variable domain of SEQ ID NO:38.

7. The pharmaceutical composition according to claim 1, characterized in that the peptide is selected from the group consisting of:
(SEQ ID NO: 26)
Ac-IK-Pqa-RHYLNWVTRQ(N-methyl)RY;
(SEQ ID NO: 32)
GIGAVLKVLTTGLPALISWIKRKRQQ;
(SEQ ID NO: 33)
FALLGDFFRKSKEKIGKEFKRIVQRIKDFLRNLVPRTES;
(SEQ ID NO: 34)
NKRFALLGDFFRKSKEKIGKEFKRIVQRIKDFLRNLVPR;
and
(SEQ ID NO: 35)
QHRYQQLGAGLKVLFKKTHRILRRLFNLAK.

8. A composition comprising a complex of:
a) a monospecific antibody that binds to digoxigenin, and
b) digoxigenin wherein the digoxigenin is conjugated to a peptide consisting of 5 to 60 amino acids
wherein the complex has been recovered after production.

9. The composition of claim 8, wherein the peptide comprises 10 to 50 amino acids.

10. The composition of claim 8, wherein the antibody of a) is a monoclonal antibody.

11. The composition of claim 10, wherein the antibody of a) comprises a heavy chain variable domain of SEQ ID NO 37 and a light chain variable domain of SEQ ID NO 36.

12. The composition of claim 10, wherein the antibody of a) is a humanized or human antibody.

13. The composition of claim 12, wherein the antibody of a) comprises a heavy chain variable domain of SEQ ID NO 39 and a light chain variable domain of SEQ ID NO 38.

14. The composition according to claim 8, wherein the peptide is selected from the group consisting of:
(SEQ ID NO: 26)
Ac-IK-Pqa-RHYLNWVTRQ(N-methyl)RY;
(SEQ ID NO: 32)
GIGAVLKVLTTGLPALISWIKRKRQQ;
(SEQ ID NO: 33)
FALLGDFFRKSKEKIGKEFKRIVQRIKDFLRNLVPRTES;
(SEQ ID NO: 34)
NKRFALLGDFFRKSKEKIGKEFKRIVQRIKDFLRNLVPR;
and
(SEQ ID NO: 35)
QHRYQQLGAGLKVLFKKTHRILRRLFNLAK.

15. A method of treating a disease with the pharmaceutical composition according to any one of claims 1 to 7, wherein the disease is selected from metabolic diseases, cancer, and inflammatory diseases.

16. (canceled)

17. (canceled)
